SEQUENCE LISTING (24 sequences)

SEQ ID NO: 1 - DNA, 32 bp, Escherichia Coli
gctctagacgtcagggagaggctgctcatttc

SEQ ID NO: 2 - DNA, 34 bp, Escherichia Coli
tgcactgcaggctgaacagaacccgtctttcgtc

SEQ ID NO: 3 - DNA, 34 bp, Escherichia Coli
tccactgcagggtcgatcagcaccttaccaatgc

SEQ ID NO: 4 - DNA, 32 bp, Escherichia Coli
cggaattcggatttgcatgatttgtaggccgg

SEQ ID NO: 5 - DNA, 30 bp, Escherichia Coli
gctctagagctgcttgccgcctgtagcagc

SEQ ID NO: 6 - DNA, 30 bp, Escherichia Coli
tgcactgcagccagcgggtttcaacgccga

SEQ ID NO: 7 - DNA, 30 bp, Escherichia Coli
tccactgcagtaactacccacgccgcgcgg

SEQ ID NO: 8 - DNA, 28 bp, Escherichia Coli
cggaattcgccacggcttgtcctaactg

SEQ ID NO: 9 - DNA, 32 bp, Escherichia Coli
gctctagagttcagccatattgctgatcctgg

SEQ ID NO: 10 - DNA, 31 bp, Escherichia Coli
tgcactgcagttccagcgttttgggcacagg

SEQ ID NO: 11 - DNA, 33 bp, Escherichia Coli
tgcactgcagtcgggtttgatgcgggtgaagta

SEQ ID NO: 12 - DNA, 32 bp, Escherichia Coli
cggaattcatccagcaaaccaccaagcacgac

SEQ ID NO: 13 - DNA, 31 bp, Escherichia Coli
gctctagaggtgcgccacataaagatgagcc

SEQ ID NO: 14 - DNA, 31 bp, Escherichia Coli
tgcactgcagaatatccttcaacagcttctg

SEQ ID NO: 15 - DNA, 34 bp, Escherichia Coli
tgcactgcagttacgccggaaccttcgccgtttg

SEQ ID NO: 16 - DNA, 33 bp, Escherichia Coli
cggaattccgccacatcttcaggattattaatg

SEQ ID NO: 17 - DNA, 37 bp, Escherichia Coli
gctctagagcaaaaaccagcaagcacggacacagcaa

SEQ ID NO: 18 - DNA, 38 bp, Escherichia Coli
tgcactgcagtgcatccttgtcagtcgcctttttaccg

SEQ ID NO: 19 - DNA, 38 bp, Escherichia Coli
tgcactgcagtgcagcacagccgcaacaccgaacaagc

SEQ ID NO: 20 - DNA, 34 bp, Escherichia Coli
cggaattccgctaccagtgaaacgcccatcgcct

SEQ ID NO: 21 - DNA, 20 bp, Escherichia Coli
cctgcatatatagtatgacg

SEQ ID NO: 22 - DNA, 20 bp, Escherichia Coli
tgaaagtcgtcacaaacggc

SEQ ID NO: 23 - DNA, 20 bp, Escherichia Coli
aacgtcttgctcgaggccgc

SEQ ID NO: 24 - DNA, 27 bp, Escherichia Coli
ctgcaatttattcatatcaggattatc

(19) United States
(12) Patent Application Publication
(10) Pub. No.: US 2010/0291148 A1
(43) Pub. Date: Nov. 18, 2010

(54) Shigella mutants in the functions pertaining to the processes of maturing and recycling peptidoglycans and their uses as immunogens

(76) Inventors: Maria Lina Bernardini, Roma (IT); Jean Philippe Sansonetti, Paris (FR)

Correspondence Address: LUCAS & MERCANTI, LLP, 475 PARK AVENUE SOUTH, 15TH FLOOR, NEW YORK, NY, 10016, US

(21) Appl. No.: 11/659,461

(22) PCT Filed: Aug. 4, 2005
(86) PCT No.: PCT/IT05/00472

§ 371 Date: Jun. 15, 2009

(30) Foreign Application Priority Data
Aug. 5, 2004 (IT) RM2004A000403

(51) Int. Cl. A61K 39/112; A61P 31/04; C12N 1/21

(52) U.S. Cl. 424/234.1; 435/252.3

(57) ABSTRACT

Bacteria belonging to the Shigella genus, mutated to inactivate the function of at least one of the gene products responsible for the metabolism and/or recycling of the peptidoglycans of the cellular wall, for immunogenic use, especially mutated at SltY, MltA, MltB, ampG, amiA, MppA genes, and immunogenic compositions comprising them.

### BACKGROUND

Shigellas are enteroinvasive human pathogens, which cause bacillary dissentery or shigellosis, an acute inflammation of the colon and rectal mucous membrane, responsible for 150 million cases every year, whereof 8-15% with fatal outcome (Kotloff et al., 1999). Shigellosis is a pathology that is essentially based on an uncontrolled innate immune response of the colon tissue triggered by a presence of a small number of bacteria, in the order of one hundred. In the inflammatory reaction, the following responses prevail: an excessive production of IL-8 produced by epithelial cells, a massive production of TNF-60, and a rapid secretion of IL-1β e IL-18 by the macrophages which enter into apoptosis as a result of contact with the Shigellas.

These micro-organisms invade epithelial cells, and are thus exposed to the receptor of the recognition pattern (PRR) Nod1, which recognizes specific bacterial fragments such as γ-D-glutamyl-meso-diaminopimelic (iE-DAP) acid of the bacterial peptidoglycan (PGN) of gram-negative bacteria. As a result of this recognition, Nod1 activates NF-κB (Girardin et al., 2003a) which, in turn, promotes the transcription of different genes encoding for cytokines, chemokines, adhesion molecules and enzymes involved in the elimination of the pathogen (Baeuerle and Henkel, 1994). Muramyl dipeptide (MDP) is the minimum essential structure of PGN recognized by the PRR Nod2, present in all bacteria, whose activity was prevalently described in myeloid cell lines.

The PGN molecule (murein) is constituted by glycan sheets produced starting from aminosugars, N-acetylglucosamine (GlcNAc) and N-acetylmuramic acid (MurNAc), joined together by a glycosidic bond β-1,4. The presence of a carboxylic group in muramic acid allows the formation of a covalent bond with a tetrapeptide. These linear polymers are then cross connected to each other by peptidic bonds between the terminal D-alanine of a tetrapeptide and the aminic group of lysine in position 3 of the adjacent tetrapeptide, to form a mesh that is characteristic of the structure of murein.

Although the chemistry of glycan sheets shows only some variations between different bacteria, peptides vary greatly: fundamentally, a dibasic amino acid must be present to allow the formation of an associated peptidic bond. In Escherichia coli, this is mesodiaminopimelic acid, the essential molecule that mediates the recognition of gram-negative intracellular pathogens with Nod1. The well known model of “three-for-one” proposed by Holtje in 1993 (Holtje, 1993) describes a development of the basal portion of the murein single layer in E. coli produced by a multi-enzymatic complex during bacteria growth. The enzymatic complex inserts three new sheets in the PGN layer under stress and it simultaneously removes the acceptor sheet. Since the murein synthesis machinery acts simultaneously with the machinery for its removal, it is clear that the functions relating to the synthesis of its monomers coexist with those involved in destruction.

Some polymorphisms and mutations in the nod2 gene are responsible for deregulating the innate response that leads to Crohn's pathology, indicating a possible role of bacterial PGNs and of the Nod1 and Nod2 receptors during shigellosis.

Three lytic transglycosylases (LTs) have been proposed as components of this multi-enzymatic complex and as participants in the related functions in the destruction of the PGN: SltY, MltA and MltB. The LTs enzymes similar to the lysozyme which cut the glycosidic β-1-4 bond between N-acetyl muramic acid and N-acetyl-glucosamine, favoring intramolecular transglycosylation of muramic acid (Holtje, 1995). Hence, substantially, these enzymes are catalyzing a reaction induced by intramolecular glycosyltransferase. In E. coli more or less half the PGN content is substituted at each generation, following the growth of murein and its elongation: PGP turnover is a direct consequence of the growth mechanism used by the bacteria. The resulting excess PGN is in turn modified in periplasm, transferred in cytoplasm and possibly recycled in the formation of new monomers (Park, 2001; Goodell, 1985). The priority path for withdrawal and turn-over in the cytoplasm of products generated within the periplasm takes place through the carrier protein AmpG (Jacobs et al., 1994), a transmembrane which appears to act as a specific permease for intact muropeptides, which are prevalently muropeptides substituted in tripeptides and this is due to the presence of L-D-carboxypeptidase in the periplasm. In accordance with the AmpG function, E. coli mutants lacking the AmpG function release a great quantity of PGN material in the culture medium (Jacobs et al., 1994). Next to the intact muropeptides, some degradation products of the turnover of the PGN material are captured by the cells. The presence of amidase, AmiA (Van Heijenoort et al., 1975) in the periplasm causes the degradation of the turnover products, dividing peptides from sugars. Peptides are withdrawn by the cell through a peptidic transport system, promoted by oligopeptidic (Opp) permease (Park et al., 1998) as a result of the interaction between the muropeptides and the permease specific for peptidic murein, MppA.

Salmonella is known to remodel the structure of its PGNs when it resides in macrophages (Quintela et al., 1997), probably to adapt itself to its intracellular life. Similarly, we have found that the gene that encodes for the LT, S1tY is expressed selectively and to a greater extent by Shigella during the intracellular stage of the invasive process (Bartoleschi et al., 2002), strongly suggesting that the LT play a role in the adaptation of Shigella in the conditions met within the host. The LT, which normally act as pacemakers promoting the widening of the murein, are potentially autolytic enzymes and their activity appears to be closely controlled by the cell (Holtje, 1995). They are commonly called “smart enzymes” for the precise time control of their activity: every dysfunction can lead to bacterial autolysis.

The authors' observation on the functions of the sitY gene of Shigella and of PGN, which does not have only a role in maintaining microorganism growth but is also a target recognition structure, important for the immune response, were the rational elements that induced them to manipulate this Shigella structure. The experimental design in the construction of the altered mutant in the Shigella peptidoglycan consists of the association or the presence of two classes of mutations: in LTs and in the functions involved in PGN recycling.

The genes selected as targets for mutagenesis include three LTs encoding genes, S1tY, M1tA and M1tB, and three genes encoding enzymes that play a key role in the recycling process, ampG, which encodes for the muropeptidic carrier AmpG, amiA, which encodes for muramyl-L-alanyl amidase A, and mppA, which encodes for the MppA protein responsible for transferring muropeptides from the periplasm to the cytoplasm. The combination of these two classes of mutation in a single strain generates strains altered in the PGN organization—caused by the mutations in the LTs, in which the ability to recycle the PGN components is modified. The selected functions were supposed not to alter the normal growth of Shigella as it was described in the numerous mutants in the LTs of E. coli which have no defect in growth rendering (Kraft et al., 1999). The mutants in mppA and ampG in E. coli have similar growth to the wild strain (Jacobs et al., 1994; Park et al., 1998). Mutants in the Shigella PGN are presumed to be able to release in vivo considerable quantities of wild PGN components or mutagenized PGN components. For this reason they represent a unique means to study the influence of the organization of the PGN on the activation of Nod1 and Nod2, and at an operational level, to manipulate the immune response of the host as a result of Shigella infection.

Muramyl dipeptide (MDP), the motif of PGN recognized by the Pathogen Recognition Receptor (PRR) Nod2, is known to be an adjuvant that stimulates the generation of antigen-specific T and B-lymphocyte responses and antibody production (Takada H. & Kotani S., 1995). Similarly, the dipeptide γ-D-glutamyl-meso-diaminopimelic acid (IE-DAP) contained in Gram-negative bacteria is recognized by the PRR Nod1 and has been reported to confer adjuvant activity in animal models (Takada H. & Kotani S., 1995). These results may be explained in part by the fact that MDP induces the expression of co-stimulatory molecules such as CD40, CD80 and CD86 in monocytes and dendritic cells, which mediate differentiation of naive T cells into effector T cells (Nau G. J. et al., 2002; Todate A. et al., 2001; Heinzelmann M. et al., 2000). These data emphasize the role of PGN-related molecules in contributing to the switch from innate to adaptive immune response. Furthermore, several recent studies have emphasized the role of many bacterial structures known as pathogen associated molecular patterns (PAMPs) as vaccine adjuvants (Kaisho T. & Akira S., 2002; Jiang Z. H. & Koganty R. R., 2003). Even though we haven't yet data about the potential use of Shigella mutants as a source of adjuvant or about the use of purified PGN from the Shigella mutants as adjuvants it must be recalled that the majority of strains we constructed are a natural source of vaccine adjuvant as they naturally release either wild type PGN segments or mutated PGN segments.

### DESCRIPTION OF THE INVENTION

The authors of the invention have found that upon introduction of Shigellas into host epithelial cells, bacteria activate an endogenous gene coding for a soluble lytic transglycolase (sLT), SltY, with a key role in PGN maturing and elongation, suggesting that PGN remodeling is relevant for pathogenicity. An effect of maturation and elongation of PGN consists of releasing a certain amount of “discarded” material, which is mostly internalized in the bacterial cytoplasm and subsequently recycled in units of the new PGN. From them, a small part (4-5%) is released by the bacteria and probably recognized by the PRRs Nod1 and Nod2 in the host tissues. Considering the role played by PGN fragments in the interaction between the Shigellas and the innate immune system, the authors have verified whether alterations of Shigella's PGNs are responsible for an attenuation of virulence and/or for a modulation of the inflammatory potential. To obtain an alteration of PGN elongation and/or recycling, several genes were mutagenized, including three genes that encode for LTs, SltY, MltA and MltB, and three genes that produce the enzymes responsible for PGN recycling, i.e. ampG, which encodes for a carrier for the AmpG (muro) peptide, amiA, which encodes for muramyl-L-alanyl amidase A, and mppA, which encodes for the MppA protein, responsible for transferring (muro)peptides from the periplasm to the cytoplasm. The obtainment of single, double and/or triple mutants gave rise to bacterial strains altered in the PGN organization, as in the case of LTs, and/or with an altered ability to recycle PGN components. The mutant Shigella strains so obtained are able to over-secrete PGN material, or to have an altered PGN organization with respect to the wild strain.

Following this strategy, the authors have obtained 22 strains of S. flexneri 5. Six mutants carry individual mutations (amiA, ampG, mppA, sltY, mltA, mltB), thirteen mutants have a double mutation (amiAampG, amiAmppA, amiAmltA, amiAsltY, ampGmppA, ampGmltB, ampGsltY, sltYmltA, mltBmltA, mltBsltY, mppAmltA, mppAmltB, mppAsltY) and three have a triple mutation (mltBmltAsltY, mltBsltYampG, mppAmltAsltY). The strains were analyzed for their virulence in single-layer cellular cultures in vitro and in different animal models in vivo, in the Sereny test in Guinea pigs, or the intranasal and intravenous model of infection in mice. Four mutants were also tested in the rabbit ileal loop model and in the mouse intragastric and intravenous infection model. The expert will realize that any other group of Shigella may be used (i.e. group 2, group 6, etc.), as well as other Shigella species (i.e., dysenteriae, sonnei, etc.).

Data show that mutations do not compromise growth ability. Equally, most of these mutants exhibits no difference in invasive ability with respective to the wild strain in vitro. On the contrary, virulence and/or inflammatory potential in vivo are enormously modulated by alterations in the PGN.

The attenuation of some of these mutants combined with a selective immunogenicity makes them advantageous as vaccine candidates. Results on immunization show that the strains have a better immunogenic potential than the wild type.

Therefore, the object of the present invention is a bacterium belonging to the Shigella genus, mutated to inactivate the function of at least one of the gene products responsible for the metabolism and/or recycling of peptidoglycans of the cell wall for immunogenic use. Preferably, the inactivated gene products are lytic transglycosylase enzymes, more preferably they are encoded by the following genes: SltY, MltA, MltB.

Alternatively, the inactivated gene products act in peptidoglycan recycling; preferably, they are encoded by the following genes: ampG, amiA, MppA.

A preferred embodiment is a bacterium belonging to the Shigella genus, mutated to inactivate the production of at least two of the gene products responsible for the metabolism and/or recycling of the peptidoglycans of the cell wall for immunogenic use.

Preferably, the two inactivated gene products are encoded by the amiA and mltA genes, or by amiA and mppA, or by amiA and sltY, or by amiA and ampG, or by mppA and mltB.

A preferred embodiment is a bacterium belonging to the Shigella genus, mutated to inactivate the production of at least three of the gene products responsible for the etabolism and/or recycling of the peptidoglycans of the cellular wall for immunogenic use.

Preferably, the three inactivated gene products are encoded by the sltY, mltA and mppA genes, or sltY, mltB and ampG, or by sltY, mltA and mltB.

According to a preferred embodiment, the bacterium belongs to the Shigella flexneri species. Alternatively, the bacterium belongs to the Shigella dysenteriae species.

Alternatively, the bacterium belongs to the Shigella sonnei species.

The expert in the field easily may obtain the bacteria of the invention departing from the proper Shigella strain and mutagenizing it according to standard recombinant DNA procedures, utilizing primers that are able to hybridize with relevant regions of the selected strain. Primers as below described are able to hybridize to all of Shigella mentioned species. Examples of Shigella strains that not limit the scope of protection are: Shigella sonnei (ATCC N. 11060), Shigella Dysenteriae (ATCC N. 49548), Shigella flexneri 2a http://www.ncbi.nlm.nih.gov/genomes/framik.cgi?db=Genome&gi=257 (ATCC N. 25875), (Wei et al.,2003), Shigella flexneri 5 (ATCC N. 9204), (Institut Pasteur Collection CIP 52.44), (Sansonetti et al., 1982, Venkatesan et al. 2001)

The expert in the field shall realize that the invention is not limited to the listed strains, in particular given the high homologies between species and to the fact the full sequence of many Shigella species DNA is available http://www.sanger.ac.uk/Projects/Escherichia_{—}Shigella/.

Another object of the invention is to provide an immunogenic composition comprising an immunogenically effective and acceptable amount of at least one of the mutated bacteria or components thereof, and/or appropriate adjuvants and/or excipients and/or dilutants. Preferably, the immunogenic composition is a vaccine for Shigella infections.

### DESCRIPTION OF THE FIGURES

FIG. 1. Densitometry of an RT-PCR experiment conducted on lungs of mice infected intranasally with a dose of 10⁸ bacteria of the aforesaid strains, 72 hours after inoculation.

FIG. 2. PGN mutants activate NF-κB in epithelial cells. NF-κB activity has been measured in HEK293 cells transfected with a plasmid (Igk)3-luciferase containing three sites κB recognized by NF-κB upstream the reporter gene lux as described by Philipott (Philipott 2000). The cells were infected with the individual strains and the luciferase activity quantified after two hours of incubation post-infection.

FIG. 3. Hematoxylin-eosin staining of tissue sections of lungs of mice infected with M90T, M90T ampG, and M90T mppA at 72 h p.i. and of uninfected control mice. The uninfected control shows a normal lung section; note the absence of inflammatory infiltrates, the thin aspect of alveolar septa, and the absence of BALT activation or perivascular cuffs. The lung section of animals infected with M90T shows severe suppurative bronchopneumonia characterized by cellular exudate filling alveolar spaces and airways in the absence of BALT activation. The samples of mice lungs infected with M90T ampG show a certain degree of bronchopneumonia characterized by some alveolar spaces with moderate degrees of PMN infiltration. In lungs infected with M90T mppA the alveolar spaces and airways are free from inflammatory cells, while areas of strong BALT activation are shown.

FIG. 4. Haematoxylin-eosin staining of tissue sections of liver of mice uninfected, infected with M90T, M90T ampG and M90T mppA at 48 h p.i. In sections of liver of mice infected with M90T severe degeneration and cloudy swelling of hepatocytes are accompanied by necrotic areas in which cellular morphology is completely altered. A microgranuloma, resulting in mononuclear cell aggregates with few, interspersed neutrophils, infiltrating into pericentrolobular tract is present. In the liver samples of animals infected with M90T ampG. moderate mononuclear cell infiltration, hepatic degeneration and an evident, although small, microgranuloma are present In sections of livers of mice infected with M90T mppA the normal structure of hepatic parenchyma characterized by the absence of necrosis is observable. A large microgranuloma, resulting in mononuclear cell aggregates is evident

### MATERIALS AND METHODS

Conditions of growth of the bacterial strains. The bacterial strains used in this study are listed in Table 1. The bacteria are cultivated in triptone-soy culture medium (TSB) (BBL, Becton Dickinson & Co., Cockeysville, Md.) in broth or with the addition of agar (Miller, 1992). The bacteria's ability to bind the Congo red pigment (Crb phenotype) was tested using TSA plates containing 0.01% of Congo red dye (Cr). Hektoen enteric agar (HEA) (Oxoin Ltd., Basingstoke, Hampshire, England) was used to grow the Shigelles retrieved from the infected organs. When necessary, Kanamycin (Km), ampicillin (Ap), streptomycin (Sm) and chloramphenicol (Cm) were added to the culture means at the respective concentrations of 50, 100, 100 and 20 μg/ml.

Genetic procedures. Conjugation was achieved as described by Miller (Miller, 1992). The generalized transduction mediated by P1 was conducted as described in Cersini et al., 2003, in accordance with Miller's procedure (Miller, 1992). The transductants were initially selected according to resistance to the antibiotic. They were analyzed under the molecular profile through PCR utilizing primers external to the introduced mutation and in condition of auxotrophic growth in minimum medium.

Recombinant DNA technique. The plasmid and genomic DNA was prepared with commercial kits (Qiagen GmbH, Germany). The enzymes and the buffers for the recombinant DNA procedure were obtained from Boehringer (Indianapolis, Ind.). DNA electroporation was conducted with a gene-pulser apparatus from Bio-Rad (Hercules, Calif.). PCR products were cloned using a ligation kit from Sure Clone™ (Amersham Pharmacia Biotech) and a kit of Perfectly Blunt™ donation (Novagen, Madison, Wis.). The construction of the mutants of S. flexneri M90T ΔmltB, M90T ΔmltA, M90T ΔampG, M90T ΔmppA, M90T ΔamiA had the same experimental strategy.

To obtain the ΔmltA M90T mutant, two DNA fragments, of 452-bp and of 533-bp, of the mltA gene of S. flexneri 5a were amplified using the PCR technique, using the following primers:

(Seq.Id. 1)

mltAF1 (5′-GCTCTAGACGTCAGGGAGAGGCTGCTCATTTC-3′)

(Seq.Id. 2)
mltAR1 (5′-TGCACTGCAGGCTGAACAGAACCCGTCTTTCGTC-3′)

for the fragment 1;

(Seq.Id. 3)

mltAF2 (5′-TCCACTGCAGGGTCGATCAGCACCTTACCAATGC-3′)

(Seq.Id. 4)

mltAR2 (5′-CGGAATTCGGATTTGCATGATTTGTAGGCCGG-3′)
for the fragment 2.

Thus were also generated mutants ΔmltB M90T, starting from two DNA fragments, of 452-bp and of 520-bp, of the mltB gene of S. flexneri 5a which was amplified through PCR using the following primers:

(Seq.Id. 5)

mltBF1 (5′-GCTCTAGAGCTGCTTGCCGCCTGTAGCAGC-3′)

(Seq.Id. 6)

mltBR1 (5′-TGCACTGCAGCCAGCGGGTTTCAACGCCGA-3′)
for the fragment 1;

(Seq.Id. 7)

mltBF2 (5′-TCCACTGCAGTAACTACCCACGCCGCGCGG-3′)

(Seq.Id. 8)

mltBR2 (5′-CGGAATTCGCCACGGCTTGTCCTAACTG-3′)
for the fragment 2.

For the ΔampG M90T mutant, two DNA fragments, of 575-bp and 636-bp, of the ampG gene of S. flexneri 5a, were amplified starting from the following primers:

(Seq.Id. 9)

ampGF1 (5′-GCTCTAGAGTTCAGCCATATTGCTGATCCTGG-3′)

(Seq.Id. 10)

ampGR1 (5′-TGCACTGCAGTTCCAGCGTTTTGGGCACAGG-3′)
for the fragment 1,

(Seq.Id. 11)

ampGF2 (5′-TGCACTGCAGTCGGGTTTGATGCGGGTGAAGTA-3′)

(Seq.Id. 12)

ampGR2 (5′-CGGAATTCATCCAGCAAACCACCAAGCACGAC-3′)
for the fragment 2.

For the ΔmppA M90T mutant, two DNA fragments were sequenced, of 684-bp and 498-bp, of the mppA gene of S. flexneri 5a, which were consequently amplified by the following primers:

(Seq.Id. 13)

mppAF1 (5′-GCTCTAGAGGTGCGCCACATAAAGATGAGCC-3′),

(Seq.Id. 14)

mppAR1 (5′-TGCACTGCAGAATATCCTTCAACAGCTTCTG-3′)
for the fragment 1;

(Seq.Id. 15)

mppAF2 (5′-TGCACTGCAGTTACGCCGGAACCTTCGCCGTTTG-3′)

(Seq.Id. 16)

mppAR2 (5′-CGGAATTCCGCCACATCTTCAGGATTATTAATG-3′)
for the fragment 2.

For ΔamiA M90T the primers were

amiAF1

(Seq.Id. 17)

(5′-GCTCTAGAGCAAAAACCAGCAAGCACGGACACAGCAA-3′)
and
amiAR1

(Seq.Id. 18)

(5′-TGCACTGCAGTGCATCCTTGTCAGTCGCCTTTTTACCG-3′)
for the fragment 1,
amiAF2

(Seq.Id. 19)

(5′-TGCACTGCAGTGCAGCACAGCCGCAACACCGAACAAGC-3′)
and
amiAR2

(Seq.Id. 20)

(5′-CGGAATTCCGCTACCAGTGAAACGCCCATCGCCT-3′)
for the fragment 2.

The primers contain appropriate restriction sites (underlined): XbaI for all F1 primers, PstI for all R1 and F2, and EcoRI for R2. The two fragments F1 and F2 were digested with XbaI/PstI (fragment 1) and PstI/EcoRI (fragment 2), both linked to a PstI cassette 830-bp encoding for resistance to the Cm by pGEM-CAT (A.Covacci, Chiron Vaccines, Siena, Italy), or with a cassette encoding for con resistance Km PstI of 1280-bp starting from pUC4K (Amersham Pharmacia) and cloned in the sites XbaI/EcoRI of a suicide vector pGP704 (Herrero et al. 2000) to generate the corresponding plasmids.

The fragments 1 and 2 of mltB, amiA, and mppA were linked to a cassette of Kanamycin (Km); the fragments FI and F2 of ampG, mltA and also mppA were linked to a cassette Cloramphenicol (Cm).

Primers' sequences are deduced from identical regions of the E. coli chromosome.

Primers are used in reliable and reproducible fashion to build mutants from any species of Shigella, e.g. S. flexneri, S. dysenteriae, S. sonnei. Shigella species are considered “clones” of E. coli (Lan and Reeves, 2002). Genome sequencing of S. flexneri 2a http://www.ncbi.nlm.nih.gov/genomes/framik.cgi?db=Genome&gi=257 and http://www.ncbi.nlm.nih.gov/genomes/framik.cgi?db=Genome&gi=297 and sequence comparison between S. dysenteriae, S. sonnei, E.coli 042 and E.coli E2348/69 with a coverage of at least 99.95% http://www.sanger.ac.uk/Projects/Escherichia_{—}Shigella/confirmed the above statement.

The modified plasmids pGP704 (Herrero et al. 2000) were introduced in the strain of E. coli DH5α λpir and the clones were positively selected for Ap-Cm or for Ap-Km on LB plates. The manipulated plasmids were transferred in the strain Sm10 λpir and again transferred for conjugation in the strain M90T-Sm. Transconjugants were selected for Cm-LB or for Km-LB on plate and those found sensitive to Ap were analyzed for PCR using the primers: (i) external to those used to amplify the fragments 1 and 2; (ii) within a region that includes both fragments 1 and 2; and (iii) F1 and R2 combined with the primers deriving from the Km or Cm cassettes depending on genetic manipulation.

For the CM cassette the primers were:

| | |
|---|---|
| catF: 5′-CCTGCATATATAGTATGACG-3′; | (Seq.Id. 21) |
| catR: 5′-TGAAAGTCGTCACAAACGGC-3′; | (Seq.Id. 22) |

for the Km cassette the primers were:

(Seq.Id. 23)

kanF: 5′-AACGTCTTGCTCGAGGCCGC-3′;

(Seq.Id. 24)

kanR: 5′-CTGCAATTTATTCATATCAGGATTATC-3′.

Using this strategy, the following strains were obtained: M90T ΔamiA-Km; M90T ΔampG-Cm; M90T ΔmppA-Cm; M90T ΔmppA-Km; M90T ΔmltA-Cm; M90T ΔmltB-Km.

The same mutants are also obtained with the “Lamda Red” system described in Murphy and Campellone (2003) and marketed by Gene Bridges GmbH.

Recombinant plasmids with the mutated sequences were introduced in WT Shigella strains to obtain the individual mutants. From these mutants and according to the same scheme, double and triple mutants were obtained.

Peptidoglycan Analysis

Peptidoglycans were prepared as previously described by Mengin-Lecreulx and Van Heijenoort (1985) and hydrolysed by mutanolysin (muramidase from Streptomyces globisporus). The resulting muropeptides were reduced and separated by reverse-phase HPLC as described by Glauner (1988).

Analysis of PGN Material Released in e Supernatants of Bacterial Cultures

M90T, M90T ampG, M90T mppA were growth overnight in LB medium. The cultures were then centrifuged and the pellets discarded. The supernatant of each culture medium was first filtered on 0.2 mμ filters and then reduced with a rotoevaporator. The samples were finally dried by lyophilisation. De-salting was carried out by two steps gel permeation chromatography. First, samples were applied to Toyopearl TSK-40 HW (150×3 cm, eluent 100 mM NaCl in 10 mM sodium phosphate) and the product obtained by each run was successively applied to Sephadex G-50 and finally analysed by MS spectrometry.

Mass Spectrometry

Matrix-assisted laser desorption/ionization time-of flight-mass spectrometry (MALDI-TOF-MS) was performed with a Voyager DE-PRO MALDI-TOF mass spectrometer (Applied Biosystem) in the positive ion mode at an acceleration voltage of 24 kV. One μl of the sample was mixed (directly on the metallic sample surface) with 1 μl of a 20 mg/ml solution of 2,5-dihydroxybenzoic acid (DHB, Aldrich, Steinheim, Germany) in acetonitrile/0.1 M trifluoroacetic acid 7:3. The mass spectra shown are the average of at least 50 single scans.

Culture conditions of the HeLa cells. Cells are maintained in minimum essential medium (MEM, GIBCO-BRL) implemented with 10% bovine fetal serum (HyClone Laboratories, Inc., Logan, Utah).

Invasiveness assay The invasiveness assay was performed with an MOI 100 in conditions of non confluent single layer of HeLa cells (1×10⁵/ml) with a diameter of 35 mm as described by (Cersini et al., 2003).

NF-κB activity For NF-κB-luciferase HE293 cells were plated in six-well plates at a density of 1×105 cells/ml and transfected the following day using XtremeGENEQ₂ transfection Reagent (Roche, Indianapolis, Ind.) as recommended by the manufacturer, with 0.5 μg of either the NF-κB luciferase reporter gene (Igκ-luciferase) plasmid (Dikstein, et al. 1996) or the vector as reported (Philpott, et al., 2000). The transfected cells were infected with either the wild type strain or the mutants using the standard procedure. Luciferase-NF-κB-dependent activity was measured at 2 hrs post-infection using Luciferase assay system (Promega, Wis.) as suggested by the manufacturers. Uninfected cells transfected as above or transfected with the vector alone were used as controls.

NF-κB-dependent luciferase assays were carried out in duplicate and repeated at least four times. Data reported are mean and are expressed as fold of activation compared to vector expressing cells.

Enzyme-linked immunosorbent assay IgG antibodies against S. flexneri 5 LPS in sera of uninfected and immunized animals, were measured by enzyme-linked immunosorbent assay (ELISA) using a goat anti-mouse IgG (Sigma, St. Louis, Mo.) conjugate. The starting dilutions of sera was 1:25. The final dilution considered positive had an OD value that was higher than 2 standard deviations above the mean of OD values obtained from 12 unimmunized mice at the starting dilution (cutoff OD value). A postexposure titer fourfold above the mean titer of the 12 unimmunized mice was considered evidence of a significant seroconversion.

Lysis plaque assay Lysis plaques were obtained as described at one MOI 1, 10, 100 (Cersini et al., 2003).

Sereny's Test. The keratoconjunctivitis test in Guinea pigs was conducted as described using two concentrations, 10⁸ and 10⁹ CFU. Keratoconjunctivitis intensity was analyzed based on illness development time and severity, with the following scores: 0, no pathology; 1, mild conjunctivitis; 2, keratoconjunctivitis without purulence; 3, complete development of a keratoconjunctivitis with purulence (Sereny, 1957; Mallett et al., 1993).

Rabbit ileal loop assay. New Zealand rabbits weighing 2.5-3 kg (Charles River Breeding Laboratories, Wilmington, Mass.) were used for the experimental infections. From each of these animals were drawn nine intestinal loops, each 5 cm long, which were subsequently prepared as recently described (D'Hauteville et al., 2002). Inside each loop were injected 10⁹ bacteria in an isotonic solution of 0.5 ml. After eight hours of infection, the animals were sacrificed. The loops were dissected, opened longitudinally, and fixed in formaline buffered at 4% or in a zinc sulfate buffer before histopathological analysis.

Infection in Mice

5-week old female Balb/C mice (Charles River, Italy) were used in all experiments. The animals were housed in 5 or 10 per cage depending on the experiment and inspected daily.

Intranasal infections. The mice were anesthetized with an intramuscular injection of 50 μl of a solution containing Zoletil (1 mg) (Virbac Carros, France) and Xilor (2%) (BIO 985, San Lazzaro, BO, Italy) and inoculated intranasally by releasing drops of a solution of 20 μl containing 10⁸ CFU for each strain re-suspended in a 0.9% NaCl solution. After 72 hours the mice were sacrificed by cervical dislocation and the lungs were removed and processed for histopathology, bacterial count and RT-PCR analysis. Bacterial count was conducted on samples drawn, placed in a saline buffer solution of 5-ml and immediately in ice. Then, the samples were homogenized with an Ultraturrax apparatus (Ultra Turrax IKA T18 Basic, Janke and Kunkel, GmbH and Co., Staufen, Germany). Serial dilutions of the resulting solutions were plated in plates containing agar supplemented with the appropriate antibiotic and Congo red. The lungs processed for RT-PCR were removed after extensively washing with saline solution through the right atrium of the heart and they were immediately frozen in liquid nitrogen N₂. Ten mice were used per group and each experiment was repeated twice.

Intravenous infections. The mice were tested with an injection into the caudal vein of 200 μl of bacterial suspension for each experiment (BD Microlance 3, 0.4×19, Nr.20) and deaths were recorded for 4 consecutive days. Each experiment was repeated at least three times. Non infected mice received 200 μl of SSS through the caudal vein and were used as control for each experiment. At the desired time, the animals were sacrificed by cervical dislocation. To recover the shigellae from the infected tissues, the abdominal cavity was opened aseptically and the spleen and liver were removed and treated for the histological preparations, for RT-PCR studies and for bacterial count.

For the bacterial count, the tissues were placed in sterile tubes with a cold solution of SSS (5 ml for the liver and 2 ml for the spleen) and kept in ice before every other manipulation. The tissues were then homogenized (Ultra Turrax IKA T18 Basic, Janke and Kunkel, GmbH and Co., Staufen, Germany) and the serial dilutions were plated in HEA. Bacterial counts were normalized through the dilution factor and reported as CFU per organ.

Intragastric infections. The mice received sterile food and sterile water for seven days. Two days before the infection, the mice received 5 g/l of a solution containing streptomycin and were then inoculated intragastrically with 100 μl of a bacterial suspension containing 10⁸ CFU of S. flexneri 5a in a 1.4% of sodium bicarbonate. The antibiotic treatment was maintained until the end of the experiment. Intragastric inoculations were performed with a polyethylene feeding tube (Intramedic polyethylene tubing 0.38 mm×1.09 mm×3 mm) inside the stomach. Bacterial suspensions were prepared as described above. Mice were stabulated for at least 6 hours before the inoculation.

The feces of the mice were recovered daily and examined to verify the presence of shigellae. Briefly, a pool of feces of 5 mice for each cage was homogenized in 5 ml of SSS and serially diluted and then plated in TSA-Sm. Bacterial counts were normalized as a function of the dilution factor and as a function of the weight of the specimen (5 feces weighed approximately 0.2 g) and reported as CFU per gram of feces. When shigellae in the feces were less than 10³ CFU per gram of feces (which corresponds approximately to 10² CFU/feces), persistence was considered concluded.

At 72 hours and at 120 hours post-infection a certain number of animals, depending on the experiment, was sacrificed by cervical dislocation. The abdominal cavity was opened aseptically and the desired organs were removed (liver, spleen, small intestine, colon and cecum) and prepared for histopathology studies, RT-PCR analysis and the count of the recovered bacteria. The sections of tissues deriving from the bacterial count were placed in sterile tubes containing various volumes of a cold solution of SSS (5 ml for the liver, 4 ml for the colon and the small intestine, 2 ml for the spleen and cecum) and kept in ice before every other manipulation. Tissues processed for RT-PCR were removed and immediately frozen in liquid nitrogen N₂. The tissues described above were homogenized (Ultra Turrax IKA T18 Basic, Janke and Kunkel, GmbH and Co., Staufen, Germany) and the serial dilutions were plated in HEA. The bacterial counts were normalized with respect to the dilution factor used and reported as CFU per organ.

Adjuvant Activity of the PGN

Groups of 10 mice were i.n. pre-infected with 10⁸ CFU of either M90T amiA mppA or M90T amiA ampG or the non-invasive strain BS176 (Sansonetti et al, 1982), used as a control. After 5 days p.i. the mice were re-infected with either 10⁸ CFU of the wild type strain M90T or 10⁴ CFU of the pathogenic yeast Criptococcus neoformans.

C. neoformans strain 52 was obtained from the American Type Culture Collection (ATCC 24067). Yeast were cultured in Sabouraud dextrose broth (1% neopeptone and 2% dextrose; Difco, Detroit, Mich.) for 48-72 h until the stationary phase was reached, and mice were infected i.n. with 1-2×10 ⁴ yeast cells/ml in 50 μl of sterile PBS. Mice were monitored daily and sacrificed at different time points i.e. 72 hrs post-M90T infection or 12 days post-C. neoformans infection.

Lungs were removed and assessed for the presence of pathogenic microorganisms (Shigella or C. neoformans) and histopathological features.

Histopathology

The samples used for histological and immunohistochemical analysis were fixed in a 10% formaline buffer solution and coated with paraffin. Sections with a thickness of 3 micrometers were colored with eosin-hematoxylin (HE) for the histopathological exam. The histological exam included verification of the inflammation by means of a score related to the number of inflammatory cells analyzed (macrophages and neutrophils) at a magnitude of 4000× and is reported as the mean of the entire tissue.

In the liver of the animals infected intravenously, the neutrophils were classified as absent (score 0) with no or few sporadic single cells for high power histological field (HPF), few (score 1) for few cells (5 to 19) per HPF, normal (score 2) for several cells (20 to 49) per HPF, or numerous (score 3) for 50 cells or more per HPF. The quantity of mononuclear cells was considered normal when none or few of these cells were observed per HPF (score 0). The increase in the number of cells was considered per specimen with some cells per HPF (score=1), moderate (score=2) for many cells per HPF, or severe (score=3) for numerous cells per HPF. Moreover, changes in the tissue pattern (i.e. turbid swellings of hepatocytes and areas of degeneration or necrosis) as well as spots of parenchymal and/or perivascular inflammation of cellular aggregates were recorded and expressed with similar scores. The microgranuloma was defined as an aggregation of well circumscribed cells comprising 5 or more mononucleated phagocytes.

The histological exam of the intestinal mucous membrane (mouse and rabbit) included the verification of scores pertaining to the inflammation as a function of the number of inflammatory cells present (mononucleated cells like lymphocytes, plasma cells, macrophages, neutrophils, verified at a magnitude of 400× HPF). The number of lymphocyte aggregates was established according to the state of “activation” of these follicles and it was reported as a score as a function of the measure of the areas of 10 different samples selected randomly, among the follicles of the control mice and the follicles of the infected mice. Based on these evaluations, the mean non-activated area or the tenuously activated lymphoid follicles were 0.1730925 mm² (±0.02129037) (score 0 and 1), moderately activated follicles 0.32502925 mm² (±0.0422538025) (score 2), and numerously expanded follicles 2.07240047 mm² (±0.30578936667) (score 3).

The number of inflammatory cells was recorded as indicated above. In particular, the infiltration of neutrophils was classified as stated above where the number of mononucleated cells was considered normal if no or few cells per HPF were present in the intestinal glands (score 0). An increase in the number of cells was considered slight for specimens where numerous cells were observed per HPF (score 1), moderate (score 2) for specimens with multiple cells per HPF, or marked (score 3) and severe (score 4) for specimens with numerous cells ore very numerous cells per BPF, respectively.

Histological criteria for a normal colon mucous membrane included the detection of no or few mononucleated cells scattered throughout the chorion per HPF, absence of lymphoid aggregates, or no or few neutrophils intercepted within the intestinal epithelium.

Similar criteria were used for the histological exam of the liver in which the tissue changes were evaluated (i.e. turbid swellings of hepatocytes and areas of degeneration or necrosis) as well as spots of aggregates of inflammatory cells at the parenchymal and/or perivascular level.

For analysis in the lung, the number of inflammatory cells was evaluated as described recently (Cersini et al., 2003).

The histological criteria for the characterization of the normal lung tissue included the detection of no or few mononucleated cells per HPF, or no or few neutrophils found in the bronchioles and in the alveoli without any tissue change (no interstitial thickening or BALT activation or presence of exudate in the free areas).

Sections of 3 mμ-thick were also processed for immunohistochemical analysis as follows: tissue's sections were treated as recently described (Cersini et al, 2003. Martino et al, 2005).The sections were placed onto pretreated slides (Bio-Optica) and dried overnight at 37° C. After being dewaxed, sections were placed in EDTA buffer, pH 9.0, and processed in a microwave oven at 650 W for two cycles of 10 min each and cooled at room temperature for 20 min. Tissue sections were then incubated overnight in a moist chamber at 4∞C with different primary antibodies, diluted 1:50 in Tris-buffered solution (TBS) containing 0.1% crystalline bovine serum albumin (BSA). Binding of mAb was revealed with ABC-peroxydase or ABCalkaline phosphatase techniques using 1:200 diluted biotinylated conjugated rabbit anti-rat immunoglobulin G (IgG; all from Vector Laboratories) and a 1:200 diluted biotinylated goat anti-mouse Ig (AO433; DAKO), applied for 45 min at room temperature, as secondary antibodies.

The enzymatic reaction was developed with 3-1-diaminobenzydine (DAB) (Sigma), VIP or Vector Blue, and Vector Red (all from Vector) as substrates, respectively, for ABC-peroxydase and ABC-alkaline phosphatase techniques. Specific primary antibodies substituted with TBS or non-immune sera were used as negative controls.

Primary antibodies employed were a murine mAb anti-S. flexneri 5a LPS (dimeric IgA, 6 mg/ml; Cersini 2003), a rat anti-mouse mAb TNFα (Serotec, Oxford, UK), a rabbit polyclonal IgG anti-mouse IL-2Rα (CD25) (Santa Cruz Biotechnology), a rat anti-mouse mAb IL-6, a rat anti-mouse IFN-gamma and Bcl-2 both by Serotec and a rabbit anti-mouse mAb NF-kB p65 (Histolines laboratories).

Extraction of RNA and Analysis for RT-PCR

Total RNA drawn from tissues of interest was extracted using a Trizol solution (Invitrogen Italia, S. Giuliano Milanese, Italy), in accordance with the manufacturer's instructions. RNAse-free DNAse (Boehringer Mannheim) was used to remove the genomic DNA as described by Dilworth and McCarrey. The reverse transcription (RT) of the total RNA (1 μg) and the PCR of the cDNA was conducted using the Super-Script™ One-Step RT-PCR with the Taq Platinum (Invitrogen) in accordance with the manufacturer's instructions. Ten microliters for every 50 μl were visualized by LTV illumination after electrophoresis in gel containing 2% of agarose and 0.5 μg ml⁻¹ of ethidium bromide. The primers used for PCR for β-actin were analyzed for each individual specimen as standard internal positivity control. As a negative control, cDNA derived from PCR omitting the primers (water was the substitute) were run parallel.

The mRNAs of the cytokines were quantified using Quantity-one software (Bio-rad Laboratories) and the results were normalized as a function of the quantity of mRNA derived from β-actin. The mean value of three runs was used to estimate mRNA for individual mice.

Results

Construction of the Strains

Following the experimental approach described in the Materials and Methods section, 22 mutants of S. flexneri 5 M90T (Sansonetti et al.,1982), altered in the recycling of the PGN and of the LTs or in both processes were obtained: six mutants carry individual mutations (amiA, ampG, mppA, sltY, mltA, mltB); thirteen mutants carry double mutation (amiAampG, amiAmppA, amiAmltA, amiAsltY, ampGmppA, ampGmltB, ampGsltY, sltYmltA, mltBmltA, mltBsltY, mppA mltA, mppAmltB, mppAsltY); and three triple mutants (mltBmltAsltY, mltBsltYampG, mppAmltAsltY). All mutants were extensively analyzed from the molecular viewpoint to define gene inactivation following the experimental approaches described in the Materials and Methods section. Superimposable results are obtained with other Shigella species, in particular with the strain N. 301 of Shigella flexneri serotype 2a. The strains and their characteristics are listed in Table 1.

TABLE 1
SHIGELLA PGN MUTANTS AND THEIR RELEVANT
PHENOTYPES

| | Muropeptidic | Muropeptidic | Peptidic | Lytic | Soluble lytic |
|---|---|---|---|---|---|
| STRAINS | Amidase | Carrier | Carrier | transglycosylase | transglycosylase |
| amiA | • | | | | |
| ampG | | • | | | |
| mppA | | | • | | |
| mltA | | | | • | |
| mltB | | | | • | |
| sltY | | | | | • |
| amiAampG | • | • | | | |
| amiAmppA | • | | • | | |
| amiAmltA | • | | | • | |
| amiAsltY | • | | | | • |
| ampGmppA | | • | • | | |
| ampGmltB | | • | | • | |
| ampGsltY | | • | | | • |
| sltYmltA | | | | • | • |
| mltBmltA | | | | •• | |
| mltBsltY | | | | • | • |
| mppAmltA | | | • | • | |
| mppAmltB | | | • | • | |
| mppAsltY | | | • | | • |
| mltBmltAsltY | | | | •• | • |
| mltBsltYampG | | • | | • | • |
| mppAmltAsltY | | | • | • | • |

As expected, Shigella mutants grow with the same rapidity as the wild strain and show the same LPS profile for Western blot analysis using an anti-S. flexneri 5 polyclonal serum.

Peptidoglycan Analysis of Mutants

The assumption of this experimental approach was that (i) the inactivation of genes encoding for transglycoylase sltY, mltA and mltB could alter PGN organization of Shigella while the inactivation of genes encoding functions related to PGN recycling could provoke release of peptidoglycan components by Shigella.

Biochemical analysis of PGN composition of the mutants M90T sltY and M90T mltB confirm this assumption as the PGN of these strains presents different amounts of basic PGN components as shown in table 2.

Moreover, preliminary analysis of the PGN content present in growth media of M90T ampG and M90T mppA confirms that M90T ampG release at least 40% more PGN related-compounds (mainly the basic motif GlcNAc-AnhMurNAc-Ala-Glu-DAP and anhMurNAc-Ala-Glu-DAP) while M90T mppA shed essentially (muro)peptdides mainly Ala-Glu-DAP-Ala and Ala-Glu-DAP.

Therefore, on this basis we may consider that the association of these two class of mutation, those involving genes encoding transglycosylases and those impairing PGN recycling, shall lead Shigella to release different “mixture” of PGN products able to interact with the innate immune system.

TABLE 2
MUROPEPTIDE COMPOSITION OF SHIGELLA FLEXNERI
LYTIC TRANSGLYCOSYLASE MUTANTS

| | M90T (%) | M90TΔmltB (%) | M90TΔsltY (%) |
|---|---|---|---|
| Monomer tri | 4.1 | 5 (+22%) | 5.7 (+39%) |
| Tetra gly₄ | 1.4 | 1.2 | 1.3 |
| Monomer tetra | 46.8 | 41.1 (−12%) | 39.4 (−24%) |
| Monomer di | 1.8 | 2.8 | 2.8 |
| Tri Lys Arg | 2.95 | 3.4 | 3.0 |
| Dimer tetra tri | 4.2 | 5.3 (+28%) | 5.3 |
| Dimer tetra | 29.2 | 29.5 | 29.5 |
| Monomer anhydro | 1 | 1.1 | 1.0 |
| Tri tri A₂pm Lys Arg | 1 | 1.1 | 1.5 |
| Tetra tri Lys Arg | 1.2 | 1.5 | 2.0 |
| Trimer tetra | 2.3 | 2.2 | 2.3 |
| Total 1.6 | 4.6 | 3.9 (−15%) | 4.0 (−13%) |
| AnhydroMur | | | |
| peptides | | | |

Only main fragments are taken in account.

( )* indicate % changes in mutants compared with wild type strain.

Monomer di, disaccharide dipeptide ou disaccharide L-Ala-D-Glu

Monomer tri, disaccharide tripeptide ou disaccharide L-Ala-gD-Glu-mA₂pm.

Monomer tetra, disaccharide tetrapeptide ou disaccharide L-Ala-gD-Glu-mA₂pm-D-Ala.

Tri Lys Arg, monomer tri with Lys Arg residues from lipoprotein.

Monomer anhydro, disaccaride 1,6 AnhydroMur Tetrapeptide

Tri Tri A₂pm Lys Arg, dimer

Analysis of Virulence In Vitro

The virulence of these mutants was analyzed in single layers of cells in culture. The wild strain M90T and its corresponding variant deprived of the virulence plasmid BS176 were used as controls in virulence assays.

The mutants were analyzed for their ability to invade HeLa cells. All mutants have shown that they have a completely invasive phenotype with the exception of the mutant M90T ampGamiA and M90T mppAmltB. The mutant M90T ampGamiA is not able to invade HeLa cells, whilst the mutant M90T mppAmltB is poorly invasive. The percentage of invaded cells is significantly reduced (around 10%) with respect to the wild strain. Thereafter, the strains were analyzed in the lysis plaque assay. The plates observed in a single layer of HeLa cells following infection with Shigella were analyzed for the production of the cytopathic effect induced by pathogens on the host cells. This test covers the following phenotypes, characteristic of shigellae: (i) invaded cells, (ii) bacterial proliferation inside the cytoplasms of infected cells; (iii) the ability of the shigellae to move inside the confluent single layer passing intercellularly; (iv) the ability to move a direct/indirect death of the host cells in the last infection times. When a mutant is unable to perform at least one of these phenotypes, this induces a negative result in the lysis plaque assay. We have found that, in accordance with the invasiveness defects, the two aforementioned mutants—ΔampG amiA M90T and ΔmppA mltB M90T—were found negative for this test. ΔsltYmltA M90T and sltYmltAmltB M90T yielded negative results in the lysis plaque assay, suggesting that one of the described phenotypes was being altered. The results are shown in Table 3. An analysis of the secreted proteins TTSS indicates that the ΔsltYmltA M90T and sltYmltAmltB M90T strains show an altered profile of the secretory proteins which may be due to the inability to produce plaques.

TABLE 3
RESULTS OF THE LYSIS PLAQUE ASSAY AND
OF THE SERENY TEST

| | Lysis | |
|---|---|---|
| | plaque | Sereny |
| STRAINS | assay | test** |
| amiA | + | ++ |
| amiAmltA | + | − |
| amiAmppA | + | − |
| amiAsltY | + | − |
| ampG | + | ++ |
| ampGamiA | − | − |
| ampGmltB | + | +++ |
| ampGmppA | + | ++ |
| ampGsltY | + | ++ |
| BS176 (Sansonetti et al., 1982) | − | − |
| M90T (Sansonetti et al., 1982) | + | ++ |
| mltA | + | +++ |
| mltAmltB | + | +++ |
| mltB | + | +++ |
| mppA | + | +++ |
| mppAmltA | + | +++ |
| mppAsltY | + | ++ |
| mppA mltB | − | − |
| sltY | + | ++ |
| sltYmltA | − | − |
| sltYmltAmppA | − | − |
| sltYmltB | + | − |
| sltYmltBampG | + | − |
| sltYmltBmltA | − | − |

* + indicates that the strain yielded a positive result at all different MOI concentrations: 1, 10, 100.
*** The Sereny test was conducted at a dose of 10⁹ CFU. n = 3. The results were evaluated 72 hours after the test. The keratoconjunctivitis level was qualified on the basis of development time and severity and (if possible), of the level of disappearance of the symptoms with the following scores: 0, no pathology; +, mild conjunctivitis; ++, keratoconjunctivitis without purulence; +++, completely developed keratoconjunctivitis with purulence.

NF-κB In Vitro Activation

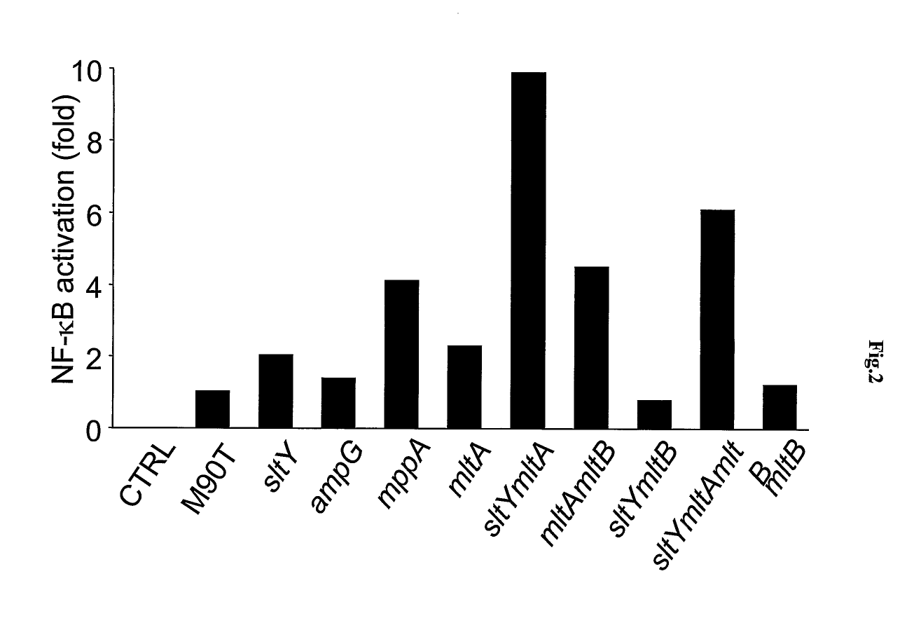

The assumption of the authors is that the Shigella mutants are able to modulate the innate immunity responses. To assess this aspect in vitro in cell lines a certain number of mutants, i.e. those impaired in lytic transglycosylases and in proteins involved in the muropeptide transport, were assessed in HEK293 cells to evaluate NF-κB activity (FIG. 2). In accordance with the mutations introduced the mutants displayed different ability to stimulate NF-κB with respect to the wild type strain. In particular, the mutants harboring sltY inactivation and M90T mppA induced high levels of NF-κB activation.

Virulence Assays In Vivo

Sereny Test

All strains were assayed in the Sereny test, a widely used virulence test based on the invasive ability of the shigellae to provoke a keratoconjunctivitis as a result of inoculation in the conjunctive of mice and Guinea pigs. The highest doses 10⁹ CFU doses, in order definitively to assert that the mutant was incapable to produce a positive result in this assay. Normally, different doses, not in a single solution were administered to the animals. (10⁸ and 10⁷ CFU) and the result was quantified according to a different score to establish the severity of the symptoms and their appearance or disappearance. For this reason, it is not possible to deduce whether these strains may have an attenuated phenotype if tested with a lower inoculation. Four double mutants related to the inactivation of the gene. amiA which encodes for muropeptidic amidase (amiAmltA, amiAmppA, amiAsltY, ampGamiA) were found to be negative. In addition to these strains, two other double mutants in two LTs, sltYmltA M90T and sltYmltB M90T yielded a negative result, as did the mutants mppAmltB M90T, which carry the activation of peptidic permease and of LT MltB. Lastly, the triple mutants were also incapable of causing keratoconjunctivitis. Table 2 shows the results of this analysis.

Intranasal and Intravenous Mouse Infections

All mutants were also analyzed through the murine shigellosis pulmonary model (Mallett et al., 1993; Phalipon et al., 1995) at the dosage of 10⁸ CFU, corresponding to 1 LD₅₀ for the wild strain. 72 hours after the infection, LD₅₀ was reached for groups of animals infected with M90T and the surviving mice were sacrificed and the lungs were removed and analyzed to evaluate several parameters: (i) the number of bacteria; (ii) the histopathological profiles; (iii) the expression of mRNA of pro-inflammatory cytokines. Table 4 shows the results obtained in this model.

TABLE 4
RESULTS OF SHIGELLOSIS IN THE MURINE PULMONARY
MODEL

| STRAINS | % of Deaths* | CFU/lung |
|---|---|---|
| amiA | 19 | 10³-10⁴ |
| amiAmltA | 14 | 10³-10⁴ |
| amiAmppA | 0 | 0-10³ |
| amiAsltY | 0 | 10²-10³ |
| ampG | 40 | 10⁵ |
| ampGamiA | 0 | 0-10³ |
| ampGmltB | 67 | 10⁶ |
| ampGmppA | 33 | 10⁵ |
| ampGsltY | 33 | 10⁵ |
| M90T | 55 | 10⁵-10⁶ |
| mltA | 27 | 10⁵ |
| mltAmltB | 36 | 10⁵ |
| mltB | 75 | 10⁵ |
| mppA | 0 | 10⁵-10⁶ |
| mppAmltA | 100 | na |
| mppAsltY | 67 | 10⁶ |
| mppAmltB | 0 | 0-10¹ |
| sltY | 40 | 10⁶ |
| sltYmltA | 20 | 10⁵ |
| sltYmltAmppA | 0 | 10³-10⁴ |
| sltYmltB | 0 | 10⁵-10⁶ |
| sltYmltBampG | 6 | 10³-10⁴ |
| sltYmltAmltB | 0 | 10² |

*The dose of the inoculum was 10⁸ CFU. n > 10. The DL50 was obtained 72 hours post infection.

Surviving animals were sacrificed at 72 hours post infection and the lungs were removed and treated for bacterial count, histopathological analysis and RT-PCR.

The strains M90T amp GamiA, M90T amiAmppA, M90T amiAsltY, M90T mltBmppA, and M90T sltYmltB do not induce death in animals infected at one LD₅₀. This group includes the four mutants found negative for the Sereny test. In accordance with the Sereny test, triple mutants were completely attenuated in this model. In contrast with the Sereny test M90T mppA did not induce death in infected animals although the number of CFUs recovered from the infected lungs was higher (10⁵) suggesting a residual virulence. The histopathology scores relating to the analysis of the infected lungs from a residual number of mutants are shown in Table 4. M90T amiA, M90T amiAmppA, M90T amiAampG stimulate a strong activation of the BALT with a recruitment of mononuclear cells in the absence of PMN.

TABLE 5
CLASSIFICATION RELATING TO THE HISTOLOGICAL EXAM
OF MURINE LUNGS INFECTED WITH SOME OF THE PGN MUTANTS

| | | Intrabronchial | | | Mononuclear | Intralveolar |
|---|---|---|---|---|---|---|
| Strains | Interstice^{a} | material^{b} | BALT^{c} | PMN^{d} | cells^{d} | desquamation |
| M90T | 3 | 4 | 2 | 4 | 2 | 4 |
| amiA | 0 | 0 | 4 | 0 | 4 | 0 |
| amiAmppA | 1 | 0 | 4 | 1 | 3 | 0 |
| ampG | 3 | 1 | 1 | 2 | 1 | 1 |
| ampGmppA | 3 | 2 | 0 | 4 | 1 | 1 |
| ampGsltY | 2 | 3 | 0 | 4 | 0 | 2 |
| mltBampG | 4 | 2 | 3 | 4 | 3 | 0 |
| mltA | 2 | 3 | 0 | 4 | 1 | 4 |
| mltAMltB | 3 | 4 | 0 | 4 | 2 | 3 |
| mppAsltY | 2 | 4 | 0 | 4 | 0 | 4 |
| sltY | 3 | 3 | 0 | 3 | 2 | 4 |
| sltYmltB | 2 | 3 | 0 | 1 | 3 | 3 |
| sltYmltBampG | 2 | 1 | 3 | 4 | 4 | 0 |
| sltYmltAmppA | 2 | 0 | 3 | 3 | 2 | 2 |

^{a}Level of thickening of the intralveolar septum due to inflammatory edema
^{b}Level of mucopurulent exudate (i.e. cell detritus, polymorphonucleated cells and proteinaceous material) observed in the airways.
^{c}Level of activation of the bronco-alveolar lymphoid tissue (i.e. presence and measurement of the free centers and of the follicle structuring of the BALT aggregates).
^{e}Level of desquamation and necrosis of the bronchial and bronchiolar epithelium.
^{d}Scores: 0 = <5 cells per HPF (high resolution level) (magnitudo 400); 1 = 5-19 cells per HPF; 2 = 20-49 cells per HPF; 3 = 50-99 cells per HPF; 4 = >100 cells per HPF.

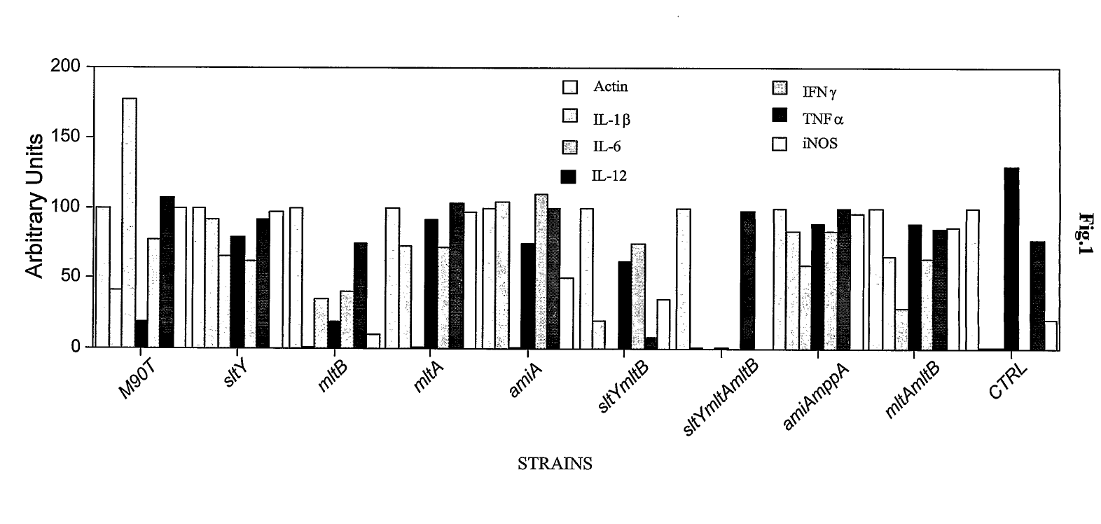

An analysis for RT-PCR conducted to evaluate the quantity of mRNA of pro-inflammatory cytokines in infected lungs showed a significant reduction in the expression of mRNA of IL-6 in all tested mutants in comparison with that of M90T and an increase in the expression of mRNA for IFN-γ in some of these, particularly M90T amiA (FIG. 1).

With a parallel approach, all mutants were analyzed through the intravenous infection model in mice. In this assay, the animals received 200 μl of shigellae directly in the caudal vein. LD₅₀ was obtained at a dose of 5×10⁶ CFU/mouse and the animals died after 48 hours post-infection of fulminating hepatitis. In this analysis, the PGN mutants were used at a dose of 10⁷ CFU, actually corresponding to 1 LD₇₀, and the surviving animals were sacrificed at 48 hours post-infection. Livers and spleens were removed and analyzed to evaluate the number of CFU and immunohistopathological damages. The results obtained in this model are shown in Table 6.

TABLE 6
RESULTS RELATING TO THE MURINE INTRAVENOUS
SHIGELLOSIS MODEL

| | | CFU | CFU | |
|---|---|---|---|---|
| | % OF | IN THE | IN THE | |
| STRAINS | DEATHS* | LIVER | SPLEEN | LD (CFU) |
| amiA | 17 | 10⁴ | 10¹ | 8 × 10⁷ |
| amiAmltA | 0 | 0 | 0 | 10⁹ |
| amiAmppA | 0 | 0 | 0 | >10⁸ |
| amiAsltY | 0 | 0 | 0 | 10⁸ |
| ampG | 83 | 10⁵ | 10² | 10⁷ |
| ampGamiA | 0 | 10³ | 10¹ | >10⁸ |
| ampGmltB | 50 | 10⁶ | 10¹ | 5 × 10⁷ |
| ampGmppA | 0 | 10⁵ | 10¹ | 10⁸ |
| ampGsltY | 50 | 10⁷ | 10³ | 5 × 10⁷ |
| BS176 | 0 | 10² | 10¹ | 10⁸ |
| M90T | 70 | 10⁴ | 10² | 3 × 10⁷ |
| mltA | 0 | 10⁶ | 10² | >5 × 10⁷ < 10⁸ |
| mltAmltB | 0 | 10⁶ | 10³ | >5 × 10⁷ < 10⁸ |
| mltB | 0 | 10³ | 10¹ | >5 × 10⁷ < 10⁸ |
| mppA | 8 | 10² | 10¹ | >5 × 10⁷ < 10⁸ |
| mppAmltA | 100 | na | na | 10⁷ |
| mppAsltY | 17 | 10⁶ | 10² | 8 × 10⁷ |
| mppAmltB | 0 | 10¹ | 10¹ | 10⁸ |
| sltY | 53 | 10⁶ | 10² | 5 × 10⁷ |
| sltYmltA | 43 | 10³ | 10¹ | 5 × 10⁷ |
| sltYmltAmppA | 0 | 10² | 0 | 10⁸ |
| sltYmltB | 66 | 10⁴ | 10² | 5 × 10⁷ |
| sltYmltBampG | 0 | 10¹ | 10¹ | 10⁹ |
| sltYmltBmltA | 0 | 10¹ | 0 | 10⁸ |

*The dose of the inoculum was 1 × 10⁷ CFU. n > 10. The DL70 was obtained 48 hours post infection. Surviving animals were sacrificed at 48 hours post infection and the liver and spleen were removed and treated for bacterial count and histopathological analysis.
** 25% mortality is obtained at a dose of 10⁹ CFU.

Several mutants do not induce death in the infected animals at this dose, although the presence of a high number of bacteria in the liver, observed in some of them, e.g. M90T mltAmltB may indicate a residual virulence. In accordance with this hypothesis, the authors have found that some of them showed an LD similar to that of M90T, whereas other strains, such as M90T amiAmltA, M90T amiAmppA, M90T amiAampG, M90T amiAsltY and the triple mutants, have a significantly greater LD than that of M90T. Table 7 show the histopathological scores obtained from the analysis of the liver and the spleen of the infected animals. In accordance with the previous results, the strains that carry a mutation in amiA for the inactivation of this gene would appear to exhibit a peculiar ability to induce an activation of the mononucleated cells.

TABLE 7
CLASSIFICATION RELATING TO THE HISTOLOGICAL
EXAM OF THE LIVER AND OF THE SPLEEN OF MICE
INFECTED WITH PGN MUTANTS OF SHIGELLA

| | Necrosis | | | | | | |
|---|---|---|---|---|---|---|---|
| | in the | | Peri- | | Mono- | | Activity in |
| STRAINS | liver | Microgranulomas | hepatitis | Neutrophils | nucleates | Degeneration | the spleen. |
| amiA | 0 | 0 | 0 | 0 | 1 | 0 | 0 |
| amiAmltA | 1 | 0 | 0 | 0 | 1 | 0 | 1 |
| amiAmppA | 0 | 1 | 0 | 0 | 1 | 0 | 0 |
| amiAsltY | 0 | 1 | 0 | 0 | 2 | 0 | 1 |
| ampG | 0 | 1 | 0 | 2 | 1 | 3 | 0 |
| ampGamiA | 0 | 2 | 0 | 0 | 2 | 0 | 2 |
| ampGmltB | 0 | 1 | 0 | 0 | 1 | 1 | 2 |
| ampGmppA | 2 | 0 | 0 | 2 | 1 | 2 | 0 |
| ampGsltY | 2 | 0 | 0 | 2 | 0 | 0 | 1 |
| BS176 | 0 | 1 | 0 | 0 | 1 | 0 | 0 |
| M90T | 1 | 3 | 1 | 4 | 1 | 4 | 3 |
| mltA | 2 | 0 | 0 | 3 | 0 | 2 | 1 |
| mltAmltB | 1 | 2 | 0 | 2 | 1 | 0 | 2 |
| mltB | — | — | — | — | — | — | — |
| mppA | 1 | 2 | 0 | 1 | 1 | 0 | 1 |
| mppA mltA | — | — | — | — | — | — | — |
| mppAsltY | 1 | 0 | 0 | 1 | 1 | 2 | 1 |
| mppAmltB | — | — | — | — | — | — | — |
| sltY | 2 | 1 | 0 | 1 | 1 | 2 | 1 |
| sltY mltA | — | — | — | — | — | — | — |
| sltYmltAmppA | 1 | 1 | 0 | 1 | 1 | 1 | 2 |
| sltYmltB | 2 | 0 | 0 | 2 | 0 | 2 | 0 |
| sltYmltBampG | 1 | 1 | 0 | 1 | 1 | 1 | 2 |
| sltYmltBmltA | 0 | 1 | 0 | 1 | 1 | 1 | 2 |

The neutrophils were classified as absent (score 0) with no or few sporadic single cells for high power field (HPF), mild (score 1) for few cells (5 to 19) per HPF, moderate (score 2) for several cells (20 to 49) per HPF, or severe (score 3) for 50 or more cells per HPF, more than 100 cells (score 4).

The quantity of mononuclear cells was considered normal when none or just a few of these cells were identified in HPF (score 0). An increase in the number of cells was considered slight for specimens with several cells per HPF (score=1), moderate (score=2) for many cells per HPF, or severe (score=3) for numerous cells per HPF.

Tissue changes were evaluated (i.e. nebulous swelling of hepatocytes or areas of degeneration and necrosis) as well as spots of aggregates of inflammatory cells at the perivascular and/or parenchymal level were recorded and expressed with similar scores. The microgranuloma was defined as well circumscribed when cellular aggregations of five or more mononucleated phagocytes occurred.

Virulence Assays In Vivo: Analysis of Rabbit Ileal Loop Model and the Murine Intragastric Infection Model.

Some mutants, M90T amiA, M90T ampG, M90T sltYmltB and M90T ampG mppA were analyzed in the murine intragastric infection model and in the rabbit ileal loop infection model (Mobassaleh et al., 1988). In the intragastric infection model, the mice were pre-treated with streptomycin and then infected intragastrically with 10⁸ CFU.

The feces of these animals were collected every day and after 72 hours a certain number of animals was sacrificed and some tissues such as the colon, the cecum and the sliver were removed and analyzed for bacterial count and for histopathological studies. The results are shown in Tables 8 and 9. Considering the number of bacteria in the colon, in the cecum and in the liver, no significant difference was observed in these strains. We have observed that the remaining number of bacteria is practically constant for every strain tested within the 7 days of analysis, independently of the mutation: after this time, at different times the persistence of bacteria in the organs follows the attenuation of virulence. In any case, we have also observed that the number of CFU in the colon and in the cecum was practically the same as the one identified in the feces. M90T amiA and M90T amiAmppA were able to persist 24 and 22 days in the feces respectively, suggesting that the introduced mutations do not render these strains able to proliferate inside the intestinal tissue. The other two mutants, M90T sltY mltB e M90T ampG, disappeared from the feces after 15 and 17 days of persistence. The histopathological results of the infected tissues confirm that these mutants stimulate a reaction prevalently characterized by the presence of mononucleated cells more than by the neutrophils as observed for M90T.

TABLE 8
PERSISTENCE OF THE MUTANTS IN THE FECES
AS A RESULT OF INTRAGASTRIC INFECTION

| | ^{a}Persistence in |
|---|---|
| Strains | the intestine (days) |
| M90T | 32 |
| amiA | 24 |
| ampG | 17 |
| amiAmppA | 22 |
| sltYmltB | 15 |
| BS176 | 15 |

N = 40 for each strain in two experiments
^{a}The animals were infected with 10⁸ CFU intragastrically

TABLE 9
HISTOPATHOLOGICAL CLASSIFICATION AS A RESULT OF
INTRAGASTRIC INFECTION IN COLON AND CECUM TISSUES

| | | | Mono- | | Local |
|---|---|---|---|---|---|
| Strains | Liver | Neutrophils | nucleates | Follicles | LPS |
| M90T | 3 | 3 | 2 | 2 | +++ |
| amiA | 0 | 0 | 4 | 3 | −− |
| ampG | 1 | 1 | 4 | 2 | −− |
| amiAmppA | 1 | 0 | 4 | 3 | −− |
| sltYmltB | 1 | 0 | 3 | 2 | −− |
| BS176 | 0 | 0 | 1 | 0 | + |

Tissue sections were examined 72 hours post-infection.

The number of inflammatory cells (mononucleated cells like lymphocytes, plasma cells, macrophagesandneutrophils) were identified at a magnitudo of 400× (high resolution power HPF).

The number of lymphocyte aggregates per specimen was established and the state of activation of these follicles was reported as measure of the areas of 10 different follicles, selected randomly, in the control mice with respect to the infected mice. Based on these evaluations, the mean non-activated or slightly activated area of the lymphoid follicles was 0.1730925 mm² (±0.02129037) (score 0 and 1), or moderately activated follicles 0.32502925 mm² (±0.0422538025) (score 2), and severely expanded follicles 2.07240047 mm² (±0.30578936667) (score 3).

The neutrophils were classified as absent (score 0) when no or few sporadic single cells for high power field (HPF) were found, mild (score 1) for few cells (5 to 19) perHPF, moderate (score 2) for several cells (20 to 49) per HPF, or severe (score 3) for 50 or more cells per HPF.

The number of mononuclear cells was considered normal when none or few of these cells were observed in each HPF in intestinal glands (score 0). An increase in the number of cells was considered slight for specimens with several cells per HPF (score 1), moderate (score 2) for numerous cells per HPF, or marked (score 3) and severe (score 4) for specimens with numerous and very numerous cells per HPF, respectively.

The histological criterion for a normal colon mucous membrane included the detection of no or few mononucleated cells scattered throughout the chorion per HPF, absence of lymphoid aggregates, and no or few neutrophils identified through the intestinal epithelium.

Similar criteria were used for the histological exam of the liver in which the tissue changes (i.e. nebulous thickening of hepatocytes, areas of degeneration and necrosis, microgranulomas) aggregates of inflammatory cells in spots ine pervascular and/or parenchymal area were recorded.

In the analysis of the rabbit ileal loop test, the major criterion for evaluating the strains resided in the histopathological results of the infected tissues. In spite the severe phlogosis and in some cases the necrosis of the tissue, the mutants still show a different ability from the wild strain M90T to stimulate a response of the host in which the populations of mononuclear cells prevail (Table 10).

TABLE 10
HISTOPATHOLOGICAL CLASSIFICATION OF THE ILEAL
TISSUES INFECTED WITH SHIGELLA STRAINS

| Rabbit/per Strain | Phlogosis | Necrosis | PMN | Mononucleates |
|---|---|---|---|---|
| M90T 1′ | 2 | 3 | 2 | 0 |
| M90T 2′ | 2 | 2 | 3 | 0 |
| amiA 1′ | 3 | 0 | 1 | 4 |
| amiA 2′ | 2 | 1 | 2 | 3 |
| ampG 1′ | 3 | 1 | 2 | 2 |
| ampG 2′ | 3 | 2 | 3 | 3 |
| amiAmppA1′ | 2 | 1 | 1 | 2 |
| amiAmppA 2′ | 3 | 3 | 2 | 4 |
| BS176 1′ | 2 | 0 | 3 | 1 |
| BS176 2′ | 2 | 0 | 2 | 1 |
| sltYmltB 1′ | 2 | 0 | 1 | 3 |
| sltYmltB 2′ | 2 | 1 | 2 | 3 |

The number of inflammatory cells (mononucleated cells like lymphocytes, plasma cells, macrophages and neutrophils) was established at a magnitudo of 400X (high resolution power-HPF).

The neutrophils were classified as absent (score 0) when no or few sporadic single cells per HPF were found, mild (score 1) when some cells (5 to 10) per HPF were found, moderate (score 2) when several cells (20 to 49) per HPF were found, or severe (score 3) when 50 or more cells per HPF were found.

The number of mononuclear cells was considered normal when none or just few of these cells were observed in each HPF through intestinal glands (score 0). An increase in the number of cells was considered slight for specimens with several cells per HPF (score 1), moderate (score 2) for specimens with numerous cells per HPF, or marked (score 3) and severe (score 4) for specimens with numerous and very numerous cells per HPF, respectively.

The histological criteria for a normal intestinal mucous membrane included the detection of no or just few mononucleated cells scattered throughout the chorion per HPF, and no or few neutrophils identified through the intestinal epithelium.

Characterization of the Mutants Altered in the Genes ampG and mppA.

PGN is a dynamic structure as new PGN material is inserted between pre-existing PGN strands during bacterial elongation. This process results in the cleavage of covalent bonds between muropeptides and in their release from the PGN meshwork. Following this process around 40-50% of PGN, mainly N-acetylglucosaminyl-1,6-anhydro-N-actylmuramyl-tri (tetra) peptides (GlcNAc-anhMurNac-tri-(tetra)-peptide), is released during a bacterial generation and approximately 90% of this material is recycled by bacteria. These turnover products accumulate in the periplasm, from where they are re-imported into the cytoplasm. In Escherichia coli AmpG is a transmembrane protein acting as a specific permease for intact (tri) or (tetra) muropeptides. Escherichia coli harboring the inactivated ampG shed 30-40% of the turnover products in medium growth while wild type released only 4-8% of them. Furthermore, the periplasmic binding protein MppA binds murein tripeptides and utilizes general oligopeptide permease (OppA) to transfer its bound ligand into the cytoplasm where tripeptides are recycled (for an exhaustive review, see Holtje, 1998).

The two genes ampG and mppA were chosen for a mutagenesis in S. flexneri 5 M90T in order to impair PGN recycling and to implement intact muropeptide (in the ΔampG mutant) and murein tripeptide (in the ΔmppA mutant) accumulation and possible release by shigellae to host cells and tissues.

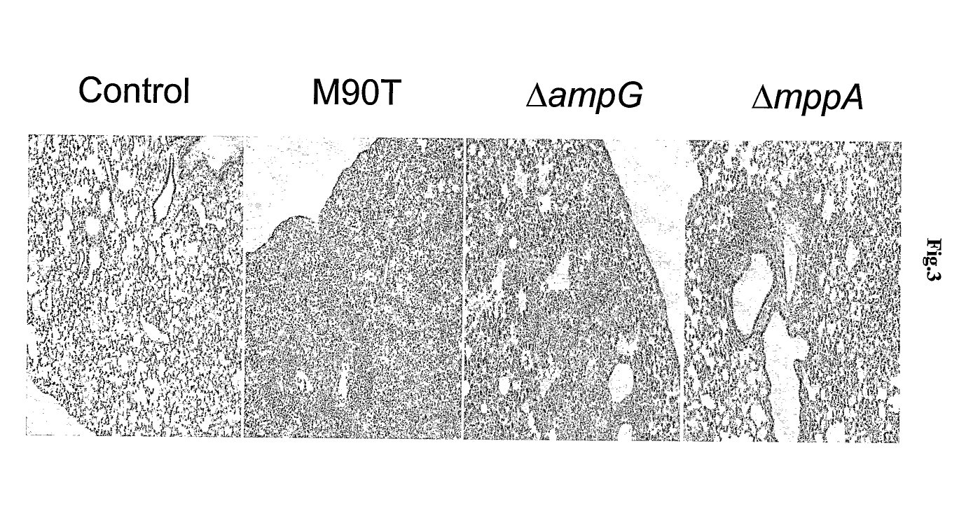

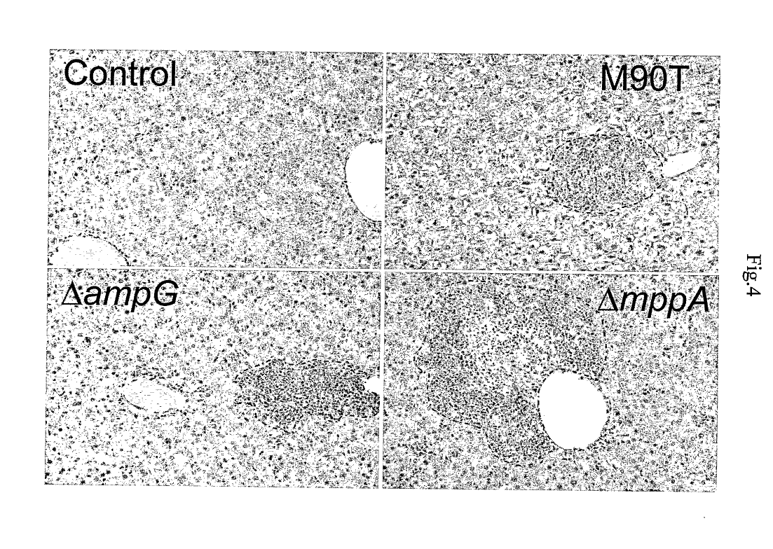

The detailed analysis of these two mutants confirmed the virulence attenuation of the strain harboring the mppA inactivation. In epithelial cells S. flexneri mppA stimulates NF-κB activation ten times more than the wild type strain (FIG. 2). In murine models, the attenuation of this strain is accompanied by the production of high level of IFN-γ and by a relevant presence of CD3/CD25 lymphocyte population, despite a residual production of IL-6 in lungs of mice infected intranasally (FIG. 3) and the production of microgranulomatous areas in the liver of mice infected intravenously (FIG. 4). CD3/CD25 cell population might reduce the severity of inflammation in the liver, while IFN-γ may contribute to the clearance of the pathogen from the infected tissues. Furthermore, in lungs BALT was clearly activated, whereas in the liver neither necrosis nor high apoptotic index was observed in the hepatic parenchyma. The results suggest that over-activation of NF-κB signaling may turn the pathological response of tissues to Shigella infection into a physiological response to this pathogen (table 11). This appears to contribute to a rapid and efficient clearance of Shigella without increasing the deleterious signs of inflammation in the infected tissues.

TABLE 11
NF-κB EXPRESSION IN THE PULMONARY TISSUE OF MICE
INFECTED WITH M90T, M90TΔampG AND M90TΔmppA
Strain

| M90T | | M90TΔampG | | M90TΔmppA | | Control | |
|---|---|---|---|---|---|---|---|
| Epithelium | MNs | Epithelium | MNs | Epithelium | MNs | Epithelium | MNs |
| 2 | 2 | 1 | 2 | 2 | 4 | 1 | 0 |

MNs mononuclear cells
Scores: 0 = <3 cells for HPF (high-power field) (400 X magnification); 1 = 3-10 cells for HPF; 2 = 11-20 cells for HPF; 3 = 21-30 cells for HPF; 4 = >31 cells for HPF.

TABLE 12
NF-κB EXPRESSION IN MICROGRANULOMATOUS
LESIONS AND IN LIVER PARENCHYMA OF ANIMALS
INFECTED WITH M90T, M90TΔampG AND M90TΔmppA

| | Within | Throughout liver |
|---|---|---|
| Strain | microgranulomas | parenchyma |
| M90T | 2 | 2 |
| M90TΔampG | 1 | 2 |
| M90TΔmppA | 4 | 3 |
| Control | — | rare |

Scores:
0 = <3 cells for HPF (high-power field) (400 X magnification);
1 = 3-10 cells for HPF;
2 = 11-20 cells for HPF;
3 = 21-30 cells for HPF;
4 = >31 cells for HPF.

TABLE 13
HISTOLOGICAL EXAMINATION OF MURINE LUNGS WITH
M90T, M90TΔampG and M90TΔmppA

| | Histological parameters | | | | | | | Immunohistochemical parameters | | | | | | | | | | | |
|---|---|---|---|---|---|---|---|---|---|---|---|---|---|---|---|---|---|---|---|
| Strain | | Intrabronchial | | | | Intralveolar | Extension of | LPS | | | | TNF-α | | | | IL-6 | | | |
| M90T | 3 | 4 | 2 | 4 | 2 | 4 | 4 | 2 | 4 | 4 | 1 | 4 | 4 | 1 | 3 | 2 | 3 | 1 | 3 |
| M90T ΔampG | 3 | 1 | 1 | 2 | 1 | 1 | 1 | 1 | 2 | 0 | 3 | 3 | 3 | 3 | 2 | 2 | 2 | 1 | 2 |
| M90T ΔmppA | 2 | 1 | 3 | 2 | 3 | 0 | 1 | 1 | 1 | 0 | 2 | 2 | 2 | 1 | 1 | 3 | 4 | 2 | 4 |
| Control | 0 | 0 | 1 | 0 | 0 | 1 | 0 | 0 | 0 | 0 | 0 | 0 | 0 | 0 | 0 | 0 | 0 | 0 | 0 |

^{a}Degree of thickening of interalveolar septa due to inflammatory oedema.
^{b}Degree of broncho and bronchiolar epithelium desquamation and necrosis.
^{c}Degree of mucopurulent exsudate (i.e. cellular debris, polimorphonuclear cells and proteinaceous material) observed in airways.
^{d}Scores: 0 = <5 cells for HPF (high-power field) (400 X magnification); 1 = 5-19 cells for HPF; 2 = 20-49 cells for HPF; 3 = 50-99 cells for HPF; 4 = >100 cells for HPF.
^{e}Degree of activation of broncho-alveolar associated lymphoid tissue (i.e. presence and size of clear centre and follicular structuration of BALT aggregates).
PMNs, plymorphonuclear leukocytes;
MNs mononuclear cells.

TABLE 14
SCORES RELATED TO HISTOLOGICAL EXAMINATION OF
THE LIVER AND THE SPLEEN OF MICE INFECTED WITH S. FLEXNERI
M90T, M90TΔampG and M90TΔmppA

| | Parameters in the liver | | | | | | | | |
|---|---|---|---|---|---|---|---|---|---|
| | Hepatic phlogosis | | | | | | | | |
| | | Mono- | Peri | | Parenchymal | Microgranulomas | | | Spleen |
| Strain | Neutrophils | nucleates | hepatitis | Degeneration | LPS | Necrosis | Mean size | LPS | activation |
| M90T | 4 | 1 | 1 | 4 | 2 | 3 | 3 | 3 | 3(*) |
| M90TΔampG | 2 | 1 | 0 | 3 | 2 | 1 | 1 | 2 | 2(*) |
| M90TΔmppA | 1 | 1 | 0 | 0 | 0 | 3 | 2 | 4 | 2(*) |
| Control | 0 | 1 | 0 | 0 | 0 | 0 | 0 | 0 | 0 |

^{a)}Number of neutrophils per HPF: score 0, less than 4 cells; score 1, 5 to 19 cells; score 2, 20 to 49 cells; score 3, 50 to 100 cells; score 4, more than 100 cells.
^{b)}Number of mononuclear cells per HPF: score 0, less than 49 cells; score 1, 50 to 100 cells; score 2, 101 to 200 cells; score 3, 201 to 600 cells; score 4, more than 600 cells
^{c)}Degree of involvement of Glissonian capsule
^{d)}Extension of necrotic areas within microgranulomas
^{e)}Mean area of granuloma extension: score 0, none; score 1, up to 0.008 square mm; score 2, up to 0.042 square mm; score 3, up to 0.4 square mm.
^{f)}Degree of LPS positivity within microgranulomas
^{g)}Degree of hepatocyte degeneration, i.e. cloudy swelling, vacuolar degeneration, hyaline and necrotising aspect with nuclear picnosis
^{h)}Degree of LPS positivity throughout the hepatic parenchyma.
ⁱ⁾Degree of white pulp activation (aspecific), mainly due to PMN cells.

TABLE 15
IHC CHARACTERIZATION OF MICROGRANULOMAS:
DISTRIBUTION OF CD3/CD25+, IFN-γ, bcl-2 POSITIVE
CELLS IN THE LIVER OF THE ANIMALS INFECTED WITH
M90T, M90TΔampG and M90TΔmppA.

| | | | | Apoptotic index | |
|---|---|---|---|---|---|
| | IHC parameters in | | | | Throughout |
| | microgranulomas | | | Within micro- | liver |
| Strain | CD3/CD25+ | IFN-γ | Bcl-2 | granulomas (a) | parenchyma (b) |
| M90T | 1 | 1 | 0 | 65.04 ± 7.21 | 15.79 ± 4.87 |
| M90T | 2 | 1 | 1 | 37.80 ± 5.59 | 13.02 ± 4.99 |
| ΔampG | | | | | |
| M90T | 3 | 2 | 3 | 17.90 ± 2.11 | 6.72 ± 2.21 |
| ΔmppA | | | | | |
| Control | 0 | 0 | 0 | — | 2.28 ± 1.12 |

Scores: 0 = <3 cells for HPF (high-power field) (400 X magnification); 1 = 3-10 cells for HPF; 2 = 11-20 cells for HPF; 3 = 21-30 cells for HPF; 4 = >31 cells for HPF.
(a) Mean apoptotic index is defined as the percentage of TUNEL-positive nuclei over the total number of cells within microgranulomas in tissue samples of liver infected with the different strains. A minimal of 10 fields per samples were analyzed at X40
(b) The value represents the mean percentage of TUNEL-positive cells calculated as above over a same number of cells in liver parenchyma outside of microgranulomas.

Immunization in Mice

To evaluate the ability of some strains to confer an immune protection against Shigella infections, we adopted the intravenous infection protocol as the immunization protocol.

The animals were immunized with an injection of 200 μl at one dose of Shigella in the caudal vein as described on day 0, 14, and 21. On day 0 and 28 the serums were collected and analyzed to evaluate protection correlates.

The immunization dose was a sub-lethal dose of the mutants of interest. On day 35 the animals were tested with the lethal dose, 5×10⁷ of the wild strain, M90T.

The surviving animals were re-tested with a lethal dose after 5 and 10 weeks, to evaluate the development of a memory of the immune protection. With a parallel approach, using the same protocol the animals were immunized with the inactivated wild bacterial strain and the inactivated bacterial strain mixed with the live mutants. The results of the protection were measured as above.

The results are shown in Tables 16 and 17.

TABLE 16
IMMUNIZATION OF THE MICE WITH LIVE BACTERIA

| | Doses | % of | % of | % of |
|---|---|---|---|---|
| Strains | (CFU) | protection | protection* | protection** |
| M90T | 10⁶ | 0 | — | — |
| amiAmppA | 10⁹ | 100 | 100 | 100 |
| amiAampG | 10⁹ | 100 | 100 | 100 |
| E. coli | 10⁹ | 0 | 0 | 0 |

N = 40 in two different experiments
*The surviving animals were re-tested with M90T LD 5 weeks later
**The surviving animals were re-tested with M90T LD 10 weeks later

TABLE 17
IMMUNIZATION OF THE MICE WITH INACTIVATED
BACTERIA

| | | | | % OF | % |
|---|---|---|---|---|---|
| STRAIN-1 | DOSE | STRAIN II | DOSE | PRO- | PRO- |
| (inactivated) | (CFU) | (live) | (CFU) | TECTION | TECTION* |
| M90T | 10⁹ | — | — | 85 | 70 |
| M90T | 10⁹ | M90T | 10⁶ | 100 | 75 |
| M90T | 10⁹ | sltY | 10⁶ | 100 | 100 |
| BS176 | 10⁹ | — | — | 73 | — |
| E. coli | 10⁹ | — | — | 0 | — |

*The surviving animals were re-tested with M90T LD 5 weeks later
N = 40 in two different experiments

Analysis of Parameters Associated with Immunization

Murine sera of animals immunized with the mutants of interest show that levels of anti-LPS IgG have a >10-fold titer increase at day 28, with strains harboring the mutations amiΔmppA and amiAampG.

The Potential use of the Mutants or of the Purified PGN as Immunomodulator.

To test whether the Shigella PGN mutated strains might acts as immunomodulators in this context the authors have applied an experimental protocol suitable to evaluate whether two strains, M90T amiA mppA and M90T amiA ampG might influence the lung microenvironment prior the exposure to pathogens.

Ten mice were therefore i.n. infected with 10⁸ CFU of these strains and with a equal dose of a non-invasive S. flexneri 5 strain, BS176, and after 5 days these animals were re-infected with the same dose (DL50) of either the wild type strain M90T or with 10⁴ CFU of the pathogenic yeast Criptococcus neoformans (Diamond 1985). Following M90T re-infection the animals were sacrificed at 72 hrs p.i. Only the animals that had received M90T amiA mppA or M90T amiA ampG seem to be protected from the infection with M90T as the mortality was considerably reduced (from 50 to 6%) and the number of bacteria in lungs was three log lower than that found in lungs of animals infected with M90T alone or pre-infected with M90T. Likewise, after 12 days of C. neoformans infection the number of yeast CFU in lungs of the animals pre-infected with M90T amiA ampG and M90T amiA mppA was three logs lower than that found in lungs of mice infected with C. neoformans alone or pre-infected with BS176. Immunohistochemical analysis confirmed that the inflammatory cell populations was significantly reduced in lungs of the mice pre-infected with these mutants.

### Conclusions

The alteration of the Shigella murein attenuates virulence but also modulates the inflammatory potential of the mutagenized strains. Therefore, a new class of Shigella mutants was isolated, in which a specific defect which resides exclusively in a modulation of the ability to induce a typical inflammatory destruction derived from shigellosis leads to the attenuation of virulence. Moreover, this approach promotes the creation of strains whose mutations can be combined differently according to the medical aims for which immunomodulation is analyzed. In vivo analysis shows that the Shigella mutants modulate the inflammatory reaction in the host, favoring the recruitment of cell populations not associated with the presence of the wild strain giving a different contribution to the activation of an adaptive immunity. Some of these strains show that they specifically activate the lymphoid tissue, bolstering this hypothesis. PGN manipulation allows to obtain strains that have a different ability to interact with Nod1 PRR which in turn activates NF-κB. In accordance with this assumption, the majority of the strains stimulates a high level of activation of NF-κB in the in vitro tests as a result of HeLa cell invasion. For some of them, the activation can be ten times greater than the one measured in the wild strain.

Two features of these strains are of relevance: (i) the extreme attenuation of many mutants, compatible with a high dosage administration; (ii) the nature of the mutations introduced, that influences bacterial recognition by the innate immune system. This aspect has been ascertained as the majority of the strains have an improved ability to stimulate the eukaryotic nuclear factor-kappa B in in vitro cultured cells such as HeLa or HEK293.

Both features might contribute to achieve a cross-protection against different Shigella species by using high doses of these strains as vaccines. This could be easily tested by using the definitive vaccine strains in trials including challenge with heterologous species.

Using the teaching of the invention it is possible to construct definitive vaccine candidates starting from main endemic Shigella species and serotypes, to be utilized as receipts to introduce the mutations. Therefore S. flexneri serotype 2a (already done), S. dysenteriae 1 and S. sonnei may be used for mutagenesis, i.e. according to Datsenko's procedure (Datsenko K. A and Wanner, B. L. 2000) that allows to obtain a ‘genetic cassette’ of mutants free of antibiotic resistance.

All in vitro assays show that this manipulation has generated mutants able, to different extents, to induce an inflammatory response in the host tissues, suggesting that the impact they produce in the host is different from the impact induced by the wild strain and is dependent upon the specific mutation.

On the other hand, a relevant attenuation combined with an increase in immunogenicity produces truly useful strains from the vaccine viewpoint.

The results of the immunization show that these mutants play a significant role in the long term increase in immunity. The analysis of the immunization correlates allow to analyze useful information on the activated immune mechanisms.

Last but not least, MDP (muramyl dipeptide) is the minimum essential structure of the bacterial PGNs required to induce biological effects, including the activity in Freund's complete adjuvant.

Innate immune cells, such as dendritic cells and macrophages, engulf pathogens by phagocytosis, and present pathogen-derived peptide antigens to naive T cells. In addition, PRRs, the Pathogen Recognition Receptor acting as the unique receptors of the innate immune system, recognize pathogen-derived components and induce expression of genes, such as co-stimulatory molecules and inflammatory cytokines. Phagocytosis-mediated antigen presentation, together with PRRs-mediated expression of co-stimulatory molecules and inflammatory cytokines, instruct development of antigen-specific adaptive immunity, especially Th1 cells (Kaisho T. & Akira S., 2002; Jiang Z. H. & Koganty R. R., 2003).

Muramyl dipeptide (MDP), the motif of PGN recognized by the (PRR) Nod2, is known to be an adjuvant that stimulates the generation of antigen-specific T and B-lymphocyte responses and antibody production (Takada H. & Kotani S., 1995). Similarly, the dipeptide γ-D-glutamyl-meso-diaminopimelic acid (IE-DAP) contained in Gram-negative bacteria is recognized by the PRR Nod1 and has been reported to confer adjuvant activity in animal models (Takada H. & Kotani S., 1995). These results may be explained in part by the fact that MDP induces the expression of co-stimulatory molecules such as CD40, CD80 and CD86 in monocytes and dendritic cells, which mediate differentiation of naive T cells into effector T cells (Nau G. J. et al., 2002; Todate A. et al., 2001; Heinzelmann M. et al., 2000). These data emphasize the role of PGN-related molecules in contributing to the switch from innate to adaptive immune response. However, the stimulatory potential of MDP alone is quite poor and it requires a vehicle, such as mineral oil, or lipid modification to enhance its activity. It is likely that these factors aid the internalisation of MDP into cells to allow interaction with NOD2. In a physiological setting, PGN may gain entry into cells through contact with bacteria over-releasing this material such as Shigella PGN mutants

On these basis the authors have performed few preliminary experiments to assess whether the mutants we have constructed might be used as immunomodulators.

It is known that prior lung immune stimulation by pathogens alters the immune response to subsequent heterologous infection (Williams et al 2004; Edwards et al, 2005). This reduces overexuberant inflammation, which can cause morbidity and mortality. Modulation of the lung microenvironment is therefore possible through exposure to this tissue to molecules, to bacteria or to bacterial material acting as immunomodulator

This experiment seems to substain the hypothesis that the PGN mutants may be used as immunomodulator material.

### BIBLIOGRAPHY

Akira, S., K. Takeda, and T. Kaisho. 2001. Toll-like receptors: critical proteins linking innate and acquired immunity. Nat. Immunol. 2: 675-680.

Baeuerle, P. A., and T. Henkel. 1994. Function and activation of NF-□B in the immune system. Annu. Rev. Immunol. 12: 141-179.

Bartoleschi C., M. C Pardini., C; Scaringi., M. C; Martino., C. Pazzani and M. L. Bernardini 2002. Selection of Shigella flexneri candidate virulence genes specifically induced in bacteria resident in host cell cytoplasm. Cell Microbiol 9:613-26

Bernardini, M. L., J. Mounier, H. d'Hauteville, M. Coquis-Rondon, and P. J. Sansonetti. 1989. Identification of icsA, a plasmid locus of Shigella flexneri which governs bacterial intra- and intercellular spread through interaction with F-actin. Proc. Acad. Sci. USA. 86: 3867-3871.

Bertin, J., W. J. Nir, C. M. Fischer, O. V. Tayber, P. R. Errada, J. R. Grant, J. J. Keilty, M. L. Gosselin, K. E. Robison, and G. H. Wong. 1999. Human CARD4 protein is a novel CED-4/Apaf-1 cell death family member that activates NF-kappaB. J. Biol. Chem. 274: 12955-12958.

Chamaillard, M., M. Hashimoto, Y. Horie, Y. Masumoto, S. Qiu, L. Saab, Y. Ogura, A. Kawasaki, K. Fukase, S. Kusumoto, M. A. Valvano, S. J. Foster, T. W. Mak, G. Nunez, and N. Inohara. 2003. An essential role for NOD1 in host recognition of bacterial peptidoglycan conyaining diaminopimelic acid. Nat. Immunol. 4: 702-707.

Cersini, A., M. C Martino, I. Martini, G. Rossi, and M. L. Bernardini, 2003 Analysis of virulence and inflammatory potential of Shigella flexneri purine biosynthesis mutants. Infect Immun 71: 1077-1089.

d'Hauteville, H., S. Khan, D. J. Maskell, A. Kussak, A. Weintraub, J. Mathison, R. J. Ulevitch, N. Wuscher, C. Parsot, and P. J. Sansonetti. 2002. Two msbB genes encoding maximal acylation of lipid A are required for invasive Shigella flexneri to mediate inflammatory rupture and destruction of the intestinal epithelium. J. Immunol. 168:5240-5251.

Diamond. R. D. 1985. Cryptococcus neoformans. In: Principles and Practice of Infectious Diseases, 2nd ed. G. L. Mandell. R. G. Doug-las, Jr., and J. E. Bennett, eds. John Wiley & Sons, New York. p. 1460.

Dikstein, R., Zhou, S., and Tjian, T. 1996 Human TAFII 105 is a cell type-specific TFIID subunit related to hTAFII130. Cell 87, 137-146

Dilworth, D. D., and J. R. McCarrey, 1992 Single step elimination of contaminating DNA prior to reverse transcriptase PCR. PCR Methods Appl 1: 279-282.

Edwards, L., Williams, A. E., Krieg, A. M., Rae, A. J., Snelgrove, R. J., and Hussell, T., 2005. Stimulation via Toll-like receptor 9 reduces Cryptococcus neoformans-induced pulmonary inflammation in an IL-12-dependent manner. Eur. J. immunol, 35 (1):273-281.

Girardin, S. E., I. G. Boneca, J. Viala, M. Chamaillard, A. Labigne, G. Thomas, D. J. Philpott, and P. J. Sansonetti. 2003b. Nod2 is a general sensor of peptidoglycan through muramyl dipeptide (MDP) detection. J. Biol. Chem. 278: 8869-8872.

Girardin, S. E., I. G. Boneca, L. A. M. Carneiro, A. Antignac, M. Jehanno, J. Viala, K. Tedin, M. K. Taha, A. Labigne, U. Zahringer, A. J. Coyle, P. S. DiStefano, J. Bertin, P. J. Sansonetti, and D. J. Philpott. 2003a. Nod1 detects a unique muropeptide from Gram-negative bacterial peptidoglycan. Science 300: 1584-1587.

Girardin, S. E., L. H. Travassos, M. Hervé, D. Blanot, I. G. Boneca, D. J. Philpott, P. J. Sansonetti, and D. Mengin-Lecreulx. 2003c. Peptidoglycan molecular requirements allowing detection by Nod1 and Nod2. Biol. Chem. J. 278: 41702-41708.

Glauner B. Separation and quantification of muropeptides with high-performance liquid chromatography Anal. Biochem. 172, 451-464 (1988).

Goodell, E. W. 1985. Recycling of murein by Escherichia coli. J. Bacteriol. 163: 305-310.

Goodell, E. W., and U. Schwarz. 1985. Release of cell wall peptides into the culture medium of exponentially growing Escherichia coli. J. Bacteriol. 162: 391-397.

Hartman, A. B., C. Powell, C. L. Schultz, E. V. Oaks, and K. H. Eckels. 1991. Small animal model to measure efficacy and immunogenicity of Shigella vaccine strains. Infect. Immun. 59:4075-4083.

Heinzelmann M., Polk H. C. Jr, Chernobelsky A., Stites T. P., Gordon L. E. 2000. Endotoxin and muramyl dipeptide modulate surface receptor expression on human mononuclear cells. Immunopharmacology 48: 117-128.

Herrero M., de Lorenzo V., and Timmis K. N. 1990. J. Bacteriol. 172, 6557-6567.

Hisamatsu T, M. Suzuki, and D. K. Podolsky 2003. Interferon-gamma augments CARD4-NOD1 gene and protein expression through interferon regulatory factor-1 in intestinal epithelial cells. J Biol Chem 278: 32962-8.

Holtje J V. 1998. Growth of the stress-bearing and shape-maintaining murein sacculus of Escherichia coli. Microbiol Mol Biol Rev 62: 181-203.

Holtje, J. V. 1995. From growth to autolysis: the murein hydrolases in Escherichia coli. Arch. Microbiol. 164: 243-254.

Hueck, C. J. 1998. Type III protein secretion systems in bacterial pathogens of animals and plants. Microbiol. Mol. Biol. Rev. 62: 379-433.

Hugot, J. P., M. Chamaillard, H. Zouali, S. Lesage, J. P. Cezard, J. Belaiche, S. Almer, C. Tysk, C. A. O'Morain, M. Gassul, V. Binder, Y. Finkel, A. Cortot, R. Modigliani, P. Laurent-Puig, C. Gower-Rousseau, J. Macry, J. F. Colombel, M. Sahbatou, and G. Thomas. 2001. Association of NOD2 leucine-rich repeat variants with susceptibility to Crohn's disease. Nature 411: 599-603.

Inohara, N., and G. Nunez. 2003. NODs: intracellular proteins involved in inflammation and apoptosis. Nat. Rev Immunol. 3: 371-382.

Inohara, N., T. Koseki, L. Del Peso, Y. Hu, C. Yee, S. Chen, R. Carrio, J. Merino, D. Liu, and J. Ni. 1999. Nod1, an Apaf-1-like activator of caspase-9 and nuclear factor-kappaB. J. Biol. Chem. 274: 14560-14567.

Inohara, N., Y. Ogura, F. F. Chen, A. Muto, and G. Nunez. 2001. Human Nod1 confers responsiveness to bacterial lipopolysaccharides. J. Biol. Chem. 276: 2551-2554.

Jacobs, C., L. J. Huang, E. Bartowsky, S. Normark, and J. T. Park. 1994. Bacterial cell wall recycling provides cytosolic muropeptides as effectors for β-lactamase induction. EMBO J. 13: 4684-4694.

Jiang Z. H. & Koganty R. R., 2003. Synthetic vaccines: the role of adjuvants in immune targeting. Curr Med Chem 10: 1423-1439.

Kaisho T. & Akira S., 2002. Toll-like receptors as adjuvant receptors. Biochim Biophys Acta 1589: 1-13.

Kotloff, K. L., J. P. Winickoff, B. Ivanoff, J. D. Clemens, D. L. Swerdlow, P. J. Sansonetti, G. K. Adak, and M. M. Levine. 1999. Global burden of Shigella infections: implications for vaccine development and implementation of control strategies. WHO Bull. 77: 651-656.

Kraft, A., J. Prabhu, A. Ursinus, and J. V. Holtje. 1997. Interference with murein turnover has no effect on growth but reduces beta-lactamase induction in Escherichia coli. J Bacteriol 181: 7192-7198

Lan and Reeves. Escherichia coli in deguise: molecular origins of Shigella Microbes and Infect 4 2002 1125-1132.

Mallett, C. P., L. VanDeVerg, H. H. Collins, and T. L. Hale. 1993. Evaluation of Shigella vaccine safety and efficacy in an intranasally challenged mouse model. Vaccine 11: 190-196.

Mandic-Mulec, I., J. Weiss, and A. Zychlinsky. 1997. Shigella flexneri is trapped in polymorphonuclear leukocite vacuoles and efficiently killed. Infect. Immun. 65: 110-115.

Martino M C, Rossi G, Martini I., Tattoli I., Chiavolini D., Phalipon A., Sansonetti P. J., Bernardini M. L., 2005. Mucosal lymphoid infiltrate dominates colonic pathological changes in murine experimental shigellosis.

J Infect Dis. 1; 192(1):136-48

Mengin-Lecreulx, D. & van Heijenoort, 1985 J. Effect of growth conditions on peptidoglycan content and cytoplasmic steps of its biosynthesis in Escherichia. J. Bacteriol. 163, 208-212.

Miller, J. H. 1992. A short course in bacterial genetics. A laboratory manual and handbook for Escherichia coli and related bacteria. Cold Spring Harbor Laboratory Press, Cold Spring Harbor, N.Y.

Medzhitov, R. 2001. Toll-like receptors and innate immunity. Nat. Rev. Immunol. 1: 135-145.

Mobassaleh, M., Donohue-Rolfe, A., Jacewicz, M., Grand, R. J., and Keusch, G. T. (1988) Pathogenesis of Shigella diarrhea: evidence for a developmentally regulated glycolipid receptor for Shigella toxin involved in the fluid secretory response of rabbit small intestine. J Infect Dis 157: 1023-1031.

Mukaida, N., Y. Mahe, and K. Matsushima. 1993. Cooperative interaction of nuclear factor κB and cis-regulatory enhancer binding protein-like factor binding elements in activation of the interleukin-8 gene by pro-inflammatoy cytokines. J. Biol. Chem. 265: 21128-21133.

Murphy K C and Campellone K G. 2003. BMC Molecular Biology 4, 11.

Nau G. J., Richmond J. F., Schlesinger A., Jennings E. G., Lander E. S., Young R. A. 2002. Human macrophage activation programs induced by bacterial pathogens. Proc Natl Acad Sci USA 99: 1503-1508.

Ogura, Y., D. K. Bonen, N. Inohara, D. L. Nicolae, F. F. Chen, R. Ramos, H. Britton, T. Moran, R. Karaliuskas, R. H. Duerr, J. P. Achkar, S. R. Brant, T. M. Bayless, B. S. Kirschner, S. B. Hanauer, G. Nunez, and J. H. Cho. 2001b. A frameshift mutation in NOD2 associated with the susceptibility to Crohn's disease. Nature 411: 603-606.

Ogura, Y., N. Inohara, A. Benito, F. F. Chen, S. Yamaoka, and G. Nunez. 2001a. Nod2, a Nod1/Apaf-1 family member that is restricted to monocyte and activates NF-kappaB. J. Biol. Chem. 276: 4812-4818.

Park, J. T., D. Raychaudhuri, H. Li, S. Normark, and D. Mengin-Lecreulx. 1998. MppA, a periplasmic binding protein essential for import of the bacterial cell wall peptide L-alnyl-γ-D-glutamyl-meso-diaminopimelate. J. Bacteriol. 180: 1215-1223.

Park, J. T. 2001. Identification of a dedicated recycling pathway for anhydro-N-acetylmuramic acid and N-acetylglucosamine derived from Escherichia coli cell wall murein. J. Bacteriol. 183: 3842-3847.

Perdomo, J. J., J. M. Cavaillon, M. Huerre, M. Ohayon, P. Gounon, and P. J. Sansonetti. 1994b. Acute inflammation causes epithelial invasion and mucosal destruction in experimental shigellosis. J. Exp. Med. 180: 1307-1319.

Perdomo, J. J., P. Gounon, and P. J. Sansonetti. 1994a. Polymorphonuclear leukocyte transmigration promotes invasion of colonic epithelial monolayer by Shigella flexneri. J. Clin. Invest. 93: 633-643.

Phalipon, A., M. Kaufmann, P. Michetti, J. M. Cavaillon, M. Huerre, P. J. Sansonetti, and J. P. Kraehenbuhl. 1995. Monoclonal immunoglobulin A antibody directed against serotype-specific epitope of Shigella flexneri lipopolysaccharide protects against murine experimental shigellosis. J. Exp. Med. 182:769-778.

Philpott, D. J., Yamaoka, S., Israel, A. & Sansonetti, P. J.,2000 Invasive Shigella flexneri activates NF-κB through a lipopolysaccharide-dependent innate intracellular response and leads to IL-8 expression in epithelial cells J. Immunol. 165, 903-914.

Quintela J. C., M. A. de Pedro, P. Zollner, G. Allmaier, and F. Garcia-del Portillo. 1997 Peptidoglycan structure of Salmonella typhimurium growing within cultured mammalian cells. Mol Microbiol 23: 693-704.

Sansonetti, P. J., A. Ryter, P. Clerc, A. T. Maurelli, and J. Mounier. 1986. Multiplication of Shigella flexneri within HeLa cells: lysis of the phagocytic vacuole and plasmid-mediated contact hemolysis. Infect. Immun. 51: 461-469.

Sansonetti, P. J., A. Phalipon, J. Arondel, K. Thirumalai., S. Banerjee, S. Akira, K, 2000. Caspase-1 activation of IL-1beta and IL-18 are essential for Shigella flexneri-induced inflammation. Immunity.; 12(5): 581-90.

Sansonetti, P. J., J. Arondel, M. Huerre, A. Harada, and K. Matsushima. 1999. Interleukin-8 controls bacterial transephitelial translocation at the cost of epithelial destruction in experimental shigellosis. Infect. Immun. 67: 1471-1480.

Sansonetti, P. J., Kopecko, D. J., and Formal, S. B. 1982 Involvement of a plasmid in the invasive ability of Shigella flexneri. Infect Immun 35: 852-860

Sereny, B. (1957) Experimental Shigella conjunctivitis. Acta Microbiol Acad Sci Hung 2: 293-296.

Takeda H. & Kotani S., 1995. In: The theory of practical application of adjuvants. Wiley, Chichester, pp. 171-202.

Takeda, and A. Zychlinsky. 2000 Caspase-1 activation of IL-1beta and IL-18 are essential for Shigella flexneri-induced inflammation. Immunity 12: 581-590.

Todate A., Suda T., Kuwata H., Chida K., Nakamura K. 2001. Muramyl dipeptide-Lys stimulates the function of human dendritic cells J Leukoc Biol 70: 723-729.

Van Heijenoort, J., C. Parquet, B. Flouret, and Y. Van Heijenoort. 1975. Envelope-bound N-acetylmuramyl-L-alanine amidase of Escherichia coli K12. Purification and properties of the enzyme. Eur. J. Biochem. 58: 611-619.

Venkatesan M. M., Goldberg M. C., Rose D. J., Burland V. and Blattner F: R:, 2001 Complete DNA sequence and analysis of the large virulence plasmid of Shigella flexneri. Infect Immun.;69(5):3271-85.

Wei J. Goldberg M B, Burland V., Venkatesan M: M:, Deng W., Fournier G., Mayhew G. F., Plunkett G., Rose D. J., Darling A., Mau B., Perna N. T., Payne S. M., Runyen-Janecky L. J., Zhou S., Schwartz D. C. and Blattner F. R.,2003. Complete genome sequence and comparative genomics of Shigella flexneri serotype 2a strain 2457T.Infect Immun.; 71(5):2775-86. Erratum in: Infect Immun. 2003 July; 71(7):4223.

Williams, A. E., Edwards L., Humphreys, I. R., Snelgrove, R., Rae, A., Rappuoli, R., Hussell, T., 2004. Innate Imprinting by the Modified Heat-Labile Toxin of Escherichia coli (LTK63) Provides Generic Protection against Lung Infectiuos Disease. The Journal of Immunology, 173: 7435-7443.

Zychlinsky, A., C. Fitting, J. N. Cavaillon, and P. J. Sansonetti. 1994. Inteleukin 1 is released by macrophages during apoptosis induced by Shigella flexneri. J. Clin. Invest. 94: 1328-1334.

Zychlinsky, A., M. C. Prevost, and P. J. Sansonetti. 1992. Shigella flexneri induces apoptosis in infected macrophages. Nature 358: 167-169.

1-15. (canceled)

16. A bacterium belonging to the Shigella genus, mutated to inactivate the function of at least one of the gene products responsible for the metabolism and/or recycling of the peptidoglycans of the cellular wall, for immunogenic use.

17. Bacterium as claimed in claim 16, wherein the gene products are lytic transglycosylase enzymes.

18. Bacterium as claimed in claim 17, wherein the inactivated gene product is encoded by at least one of the following genes: SltY, MltA, MltB.

19. Bacterium as claimed in claim 16, wherein the gene products act in the recycling of the peptidoglycans.

20. Bacterium as claimed in claim 19, wherein the inactivated gene product is encoded by at least one the following genes: ampG, amiA, MppA.

21. Bacterium as claimed in claim 16, mutated to inactivate the production of at least two of the gene products responsible for the metabolism and/or is recycling of the peptidoglycans of the cellular wall for immunogenic use.

22. Bacterium as claimed in claim 21, wherein the two inactivated gene products are encoded by amiA and mltA genes, or by amiA and mppa, or by amiA and sltY, or by amiA and amiG, or by mppA and mltB.

23. Bacterium as claimed in claim 21, mutated to inactivate the production of at least three of the gene products responsible for the metabolism and/or recycling of the peptidoglycans of the cellular wall for immunogenic use.

24. Bacterium as claimed in claim 23, wherein the three gene products are encoded by the sltY, mltA and mppA genes, or sltY, mltB and ampG, or by sltY, mltA and mltB.

25. Bacterium as claimed in claim 16, belonging to the Shigella flexneri species.

26. Bacterium as claimed in claim 16, belonging to the Shigella dysenteriae species.

27. Bacterium as claimed in claim 16, belonging to the Shigella sonnei species.

28. An immunogenic composition comprising an immunogenically effective and acceptable amount of at least one of the mutated bacteria as claimed in claim 16, or components thereof, and/or appropriate adjuvants and/or excipients and/or dilutants.

29. The immunogenic composition as claimed in claim 28 being a vaccine for Shigella infections.

30. The immunogenic composition as claimed in claim 28 for oral or intranasal administration.
